(19) United States
(12) Patent Application Publication
(10) Pub. No.: US 2022/0102009 A1
(43) Pub. Date: Mar. 31, 2022

(54) SYSTEMS AND METHODS FOR NUTRIGENOMICS AND NUTRIGENETIC ANALYSIS

(71) Applicant: Cipher Genetics, Inc., Seattle, WA (US)

(72) Inventors: Yael JOFFE, Cape Town (ZA); Jason Haddock, Cape Town (ZA)

(21) Appl. No.: 17/493,170

(22) Filed: Oct. 4, 2021

Related U.S. Application Data
(63) Continuation of application No. PCT/US2020/027462, filed on Apr. 9, 2020.
(60) Provisional application No. 62/833,393, filed on Apr. 12, 2019.

(51) Int. Cl. G16H 50/30; G16H 50/20; G16B 30/10

(52) U.S. Cl. CPC G16H 50/30; G16B 30/10; G16H 50/20

(57) ABSTRACT

The present disclosure provides systems and methods for facilitating nutrigenomics and nutrigenetic analysis. A method for generating a nutrigenetic profile of a subject may comprise: (a) receiving genetic information of the subject comprising nucleic acid sequences, obtained by processing a biological sample of the subject using nutrigenetic assays; (b) processing the genetic information to identify nutrigenetic aberrations; (c) identifying biological states corresponding to at least one of a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an infection pathway, a circadian rhythm pathway, a mood pathway, a memory pathway, a carbohydrate metabolism pathway, an adrenal pathway, a lipid metabolism pathway, a stress pathway, and a cardiovascular health pathway based at least in part on the nutrigenetic variants; and (d) outputting an electronic report indicative of the biological states.

### CROSS-REFERENCE

This application is a continuation of International Application No. PCT/US2020/027462, filed Apr. 9, 2020, which claims the benefit of U.S. Provisional Application No. 62/833,393, filed Apr. 12, 2019, each of which is incorporated by reference herein in its entirety.

### BACKGROUND

A large number of diseases or disorders may arise at least in part because of a genetic or nutrigenetic basis. Thus, analysis of genetic, nutrigenetic, or other types of data of subjects may provide valuable insights into disease causes and risks as well as lifestyle recommendations for a subject to manage his or her own health. However, nutrigenetic assays may generate complex datasets that are difficult to interpret and understand by an end user, such as a subject or patient.

### SUMMARY

Nutrigenomics or nutrigenetic data generated from one or more genetic assays may need to be efficiently collected, analyzed, and interpreted to understand how unique genetic instructions can determine the way a subject's body responds to dietary and environmental factors such as food, exercise, stress, and toxins. Thus, there is a need for accurate and effective reporting of nutrigenomics or nutrigenetic data that is comprehensive, clinically tested, and easy to understand, as well as translation of such nutrigenomics or nutrigenetic data into a practical plan of actionable, personalized recommendations for how a subject can positively impact his or her own health. Such accurate and effective reporting of nutrigenomics or nutrigenetic data may represent significant improvements in at least the technical fields of nutrigenomics and/or nutrigenetic data reporting, nutrigenomics and/or nutrigenetic data analysis, nutrigenomics and/or nutrigenetic counseling of subjects (e.g., patients), nutrigenomics and/or nutrigenetic data management, and clinical translation of nutrigenomics and/or nutrigenetic reports.

Although analysis of human genetic data, such as nutrigenomics data and/or nutrigenetic data, may produce significant insights toward advancing understanding of diseases and disorders, there can be concerns about accurate and effective reporting of nutrigenetic data that is comprehensive, clinically tested, and easy to understand. In addition, such nutrigenetic data may need to be translated into a practical plan of actionable, personalized recommendations for how a subject can positively impact his or her own health.

The present disclosure provides methods and systems for nutrigenomics and nutrigenetic analysis, including reporting of nutrigenetic data to a user.

In an aspect, the present disclosure provides a computer-implemented method for generating a nutrigenetic profile of a subject, the method comprising: (a) receiving genetic information of said subject, which genetic information comprises a plurality of nucleic acid sequences, wherein said genetic information is obtained by processing a biological sample obtained or derived from said subject using one or more nutrigenetic assays; (b) processing said genetic information to identify one or more nutrigenetic aberrations of said subject; (c) identifying one or more biological states corresponding to at least one of a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an infection pathway, a circadian rhythm pathway, a mood pathway, a memory pathway, a carbohydrate metabolism pathway, an adrenal pathway, a lipid metabolism pathway, a stress pathway, and a cardiovascular health pathway of said subject based at least in part on said one or more nutrigenetic aberrations identified in (b); and (d) outputting an electronic report indicative of said one or more biological states of said subject.

In some embodiments, said plurality of nucleic acid sequences comprises deoxyribonucleic acid (DNA) sequences, ribonucleic acid (RNA) sequences, or a combination thereof. In some embodiments, said biological sample is selected from the group consisting of saliva, cheek swab, blood, plasma, serum, urine, and a combination thereof.

In some embodiments, said one or more nutrigenetic assays comprise one or more of: a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk (e.g., Alzheimer's), a migraine test, a thyroid test, an eczema test, and a cancer genetics assay. In some embodiments, said one or more nutrigenetic assays comprise two or more of: a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk (e.g., Alzheimer's), a migraine test, a thyroid test, an eczema test, and a cancer genetics assay. In some embodiments, said one or more nutrigenetic assays comprise three or more a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk (e.g., Alzheimer's), a migraine test, a thyroid test, an eczema test, and a cancer genetics assay.

In some embodiments, said one or more nutrigenetic aberrations comprise one or more of: single nucleotide polymorphisms (SNPs), copy number variants (CNVs), insertions or deletions (indels), fusions, and translocations. In some embodiments, said one or more nutrigenetic aberrations comprise at least about 100 distinct nutrigenetic aberrations. In some embodiments, wherein said one or more nutrigenetic aberrations comprise one or more of the nutrigenetic variants in Table 1 or Table 4.

In some embodiments, said cellular pathway comprises one or more of: detoxification, DNA damage, inflammation, methylation, and oxidative stress. In some embodiments, said functional systems pathway comprises one or more of: blood clotting, bone health, collagen and joints, brain health, glucose and insulin, sex hormone balance, vascular health, cognitive decline, memory loss, mood disorders and behavior, female sex hormone balance, male sex hormone balance, and histamine tolerance. In some embodiments, said energy pathway comprises one or more of: adipogenesis, appetite/satiety/intake, energy expenditure, exercise response, pro-inflammatory fat, weight gain and weight loss resistance, circadian rhythm, and starch metabolism. In some embodiments, said activity pathway comprises one or more of: training response (VO2max), endurance, injury, power, recovery, flexibility and strength. In some embodiments, said nutrients pathway comprises one or more of: caffeine, salt, vitamin D, fatty acids, vitamin A, vitamin B6, vitamin B12, folate, vitamin C, vitamin E, choline, iron overload, iron status, gluten, celiac, and cannaboid metabolism. In some embodiments, said cardiovascular health pathway comprises one or more of: blood clotting, blood pressure, vascular health, and lipid metabolism.

In some embodiments, (c) further comprises identifying said one or more biological states of said subject based at least in part on one or more of: a diagnosis of a disease or disorder, a prognosis of a disease or disorder, a risk of having a disease or disorder, a treatment history of a disease or disorder, a history of previous treatment for a disease or disorder, a history of prescribed medications, a history of prescribed medical devices, age, height, weight, sex, smoking status, and one or more symptoms of said subject. In some embodiments, said one or more symptoms comprise chronic fatigue, weight loss, nausea, insomnia, or a combination thereof.

In some embodiments, the method further comprises generating a nutrigenetic health regimen for said subject based at least in part on said identified one or more nutrigenetic aberrations. In some embodiments, said nutrigenetic health regimen comprises recommendations to prevent onset of a disease or disorder, delay onset of a disease or disorder, reverse a disease or disorder, or maintain a physiological or health state of said subject. In some embodiments, said nutrigenetic health regimen comprises recommendations related to one or more of: diet, exercise, sports training, supplements, functional tests, blood tests, brain management, behavior change, skin care, environmental exposure, stress management, and mental health.

In some embodiments, said electronic report is indicative of said nutrigenetic health regimen. In some embodiments, said electronic report is presented on a user interface (e.g., graphical user interface) of an electronic device of a user. In some embodiments, said user is said subject. In some embodiments, the method further comprises transmitting said electronic report to a remote user. In some embodiments, said remote user is a clinical practitioner or a nutrigenetics counselor. In some embodiments, the method further comprises storing said electronic report on a remote server.

In some embodiments, (c) comprises processing said identified one or more nutrigenetic aberrations using a trained algorithm to identify said one or more biological states. In some embodiments, said trained algorithm comprises a supervised machine learning algorithm. In some embodiments, said supervised machine learning algorithm comprises a deep learning algorithm, a support vector machine (SVM), a neural network, or a Random Forest. In some embodiments, said trained algorithm comprises an unsupervised machine learning algorithm. In some embodiments, said unsupervised machine learning algorithm comprises a k-means clustering model or principal component analysis. In some embodiments, said trained algorithm is configured to identify said one or more biological states at an accuracy of at least about 80% over 50 independent samples.

In some embodiments, said electronic report comprises one or more graphical depictions of said one or more biological states of said subject. In some embodiments, the method further comprises using said electronic report to provide said subject with a therapeutic intervention. In some embodiments, the method further comprises using said electronic report to treat said subject.

In some embodiments, said method further comprises determining an impact score for each of said one or more nutrigenetic aberrations. In some embodiments, said impact score for a given nutrigenetic aberration of said one or more nutrigenetic aberrations is determined based at least in part on a scientific validity of said given nutrigenetic aberration or a clinical validity of said given nutrigenetic aberration. In some embodiments, said impact score for a given nutrigenetic aberration of said one or more nutrigenetic aberrations is determined based at least in part on a scientific validity of said given nutrigenetic aberration and a clinical validity of said given nutrigenetic aberration. In some embodiments, determining said impact score for a given nutrigenetic aberration of said one or more nutrigenetic aberrations based on said scientific validity comprises determining said impact score based on one or more of genotype frequency, rating study type, rating study quality, biological plausibility, and pathway of said given nutrigenetic aberration. In some embodiments, said impact score for a given nutrigenetic aberration of said one or more nutrigenetic aberrations is determined based on said scientific validity according to the rules and scores listed in Table 3. In some embodiments, determining said impact score for a given nutrigenetic aberration of said one or more nutrigenetic aberrations based on said clinical validity comprises determining said impact score based on one or more of biochemical impact of said given nutrigenetic aberration on clinical dysfunction or manifestation, existence of an intervention that modulates said biochemical impact, measurables and biomarkers of said given nutrigenetic aberration, and probability of benefit from said intervention. In some embodiments, said impact score for a given nutrigenetic aberration of said one or more nutrigenetic aberrations is determined based on said clinical validity according to the rules and scores listed in Table 4. In some embodiments, the method further comprises grouping said one or more impact scores that are part of the same metabolic pathway. In some embodiments, the method further comprises summing said groups of said one or more impact scores that are part of the same metabolic pathway. In some embodiments, the method further comprises expressing said summed impact scores as a percentage of the total pathway score to assign a pathway weighting to said metabolic pathway. In some embodiments, the method further comprises determining boundaries of biological states based on a maximum probable pathway score and a minimum probable pathway score from among a set of pathway scores. In some embodiments, the method further comprises determining boundaries of biological states using a supervised or unsupervised machine learning model. In some embodiments, said supervised machine learning model comprises a deep learning algorithm, a support vector machine (SVM), a neural network, or a Random Forest. In some embodiments, said unsupervised machine learning algorithm comprises a k-means clustering model or principal component analysis.

In another aspect, the present disclosure provides a system for generating a nutrigenetic profile of a subject, comprising: a database configured to store genetic information of said subject, which genetic information comprises a plurality of nucleic acid sequences, wherein said genetic information is obtained by processing a biological sample obtained or derived from said subject using one or more nutrigenetic assays; and one or more computer processors operatively coupled to said database, wherein said one or more computer processors are individually or collectively programmed to: (i) process said genetic information to identify one or more nutrigenetic aberrations of said subject; (ii) identify one or more biological states corresponding to at least one of a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an adrenal pathway, an infection pathway, a circadian rhythm pathway, a mood pathway, a memory pathway, a carbohydrate metabolism pathway, a lipid metabolism pathway, a stress pathway, and a cardiovascular health pathway of said subject based at least in part on said one or more nutrigenetic aberrations identified in (i); and (iii) electronically output a report indicative of said one or more biological states of said subject.

In some embodiments, said plurality of nucleic acid sequences comprises deoxyribonucleic acid (DNA) sequences, ribonucleic acid (RNA) sequences, or a combination thereof. In some embodiments, said biological sample is selected from the group consisting of saliva, cheek swab, blood, plasma, serum, urine, and a combination thereof.

In some embodiments, said one or more nutrigenetic assays comprise one or more of: a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk (e.g., Alzheimer's), a migraine test, a thyroid test, an eczema test, and a cancer genetics assay. In some embodiments, said one or more nutrigenetic assays comprise two or more of: a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk (e.g., Alzheimer's), a migraine test, a thyroid test, an eczema test, and a cancer genetics assay. In some embodiments, said one or more nutrigenetic assays comprise three or more a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk (e.g., Alzheimer's), a migraine test, a thyroid test, an eczema test, and a cancer genetics assay.

In some embodiments, said one or more nutrigenetic aberrations comprise one or more of: single nucleotide polymorphisms (SNPs), copy number variants (CNVs), insertions or deletions (indels), fusions, and translocations. In some embodiments, said one or more nutrigenetic aberrations comprise at least about 100 distinct nutrigenetic aberrations. In some embodiments, wherein said one or more nutrigenetic aberrations comprise one or more of the nutrigenetic variants in Table 1 or Table 4.

In some embodiments, said cellular pathway comprises one or more of: detoxification, DNA damage, inflammation, methylation, and oxidative stress. In some embodiments, said functional systems pathway comprises one or more of: blood clotting, bone health, collagen and joints, brain health, glucose and insulin, sex hormone balance, vascular health, cognitive decline, memory loss, mood disorders and behavior, female sex hormone balance, male sex hormone balance, and histamine tolerance. In some embodiments, said energy pathway comprises one or more of: adipogenesis, appetite/satiety/intake, energy expenditure, exercise response, pro-inflammatory fat, weight gain and weight loss resistance, circadian rhythm, and starch metabolism. In some embodiments, said activity pathway comprises one or more of: training response (VO2max), endurance, injury, power, recovery, flexibility and strength. In some embodiments, said nutrients pathway comprises one or more of: caffeine, salt, vitamin D, fatty acids, vitamin A, vitamin B6, vitamin B12, folate, vitamin C, vitamin E, choline, iron overload, iron status, gluten, celiac, and cannaboid metabolism. In some embodiments, said cardiovascular health pathway comprises one or more of: blood clotting, blood pressure, vascular health, and lipid metabolism.

In some embodiments, (ii) further comprises identifying said one or more biological states of said subject based at least in part on one or more of: a diagnosis of a disease or disorder, a prognosis of a disease or disorder, a risk of having a disease or disorder, a treatment history of a disease or disorder, a history of previous treatment for a disease or disorder, a history of prescribed medications, a history of prescribed medical devices, age, height, weight, sex, smoking status, and one or more symptoms of said subject. In some embodiments, said one or more symptoms comprise chronic fatigue, weight loss, nausea, insomnia, or a combination thereof.

In some embodiments, said one or more computer processors are individually or collectively programmed to further generate a nutrigenetic health regimen for said subject based at least in part on said identified one or more nutrigenetic aberrations. In some embodiments, said nutrigenetic health regimen comprises recommendations to prevent onset of a disease or disorder, delay onset of a disease or disorder, reverse a disease or disorder, or maintain a physiological or health state of said subject. In some embodiments, said nutrigenetic health regimen comprises recommendations related to one or more of: diet, exercise, sports training, supplements, functional tests, blood tests, brain management, behavior change, skin care, environmental exposure, stress management, and mental health.

In some embodiments, said electronic report is indicative of said nutrigenetic health regimen. In some embodiments, said electronic report is presented on a user interface (e.g., graphical user interface) of an electronic device of a user. In some embodiments, said user is said subject. In some embodiments, said one or more computer processors are individually or collectively programmed to further transmit said electronic report to a remote user. In some embodiments, said remote user is a clinical practitioner or a nutrigenetics counselor. In some embodiments, said one or more computer processors are individually or collectively programmed to further store said electronic report on a remote server.

In some embodiments, (ii) comprises processing said identified one or more nutrigenetic aberrations using a trained algorithm to identify said one or more biological states. In some embodiments, said trained algorithm comprises a supervised machine learning algorithm. In some embodiments, said supervised machine learning algorithm comprises a deep learning algorithm, a support vector machine (SVM), a neural network, or a Random Forest. In some embodiments, said trained algorithm comprises an unsupervised machine learning algorithm. In some embodiments, said unsupervised machine learning algorithm comprises a k-means clustering model or principal component analysis. In some embodiments, said trained algorithm is configured to identify said one or more biological states at an accuracy of at least about 80% over 50 independent samples.

In some embodiments, the system further comprises an electronic display operatively coupled to said one or more computer processors, wherein said electronic display comprises a user interface (e.g., graphical user interface) configured to display said report. In some embodiments, said electronic report comprises one or more graphical depictions of said one or more biological states of said subject. In some embodiments, the system further comprises a nutrigenetic module that is configured to process said biological sample obtained or derived from said subject to output said genetic information. In some embodiments, the method further comprises using said electronic report to treat said subject.

In some embodiments, said one or more computer processors are individually or collectively programmed to further determine an impact score for each of said one or more nutrigenetic aberrations. In some embodiments, said impact score for a given nutrigenetic aberration of said one or more nutrigenetic aberrations is determined based at least in part on a scientific validity of said given nutrigenetic aberration or a clinical validity of said given nutrigenetic aberration. In some embodiments, said impact score for a given nutrigenetic aberration of said one or more nutrigenetic aberrations is determined based at least in part on a scientific validity of said given nutrigenetic aberration and a clinical validity of said given nutrigenetic aberration. In some embodiments, determining said impact score for a given nutrigenetic aberration of said one or more nutrigenetic aberrations based on said scientific validity comprises determining said impact score based on one or more of genotype frequency, rating study type, rating study quality, biological plausibility, and pathway of said given nutrigenetic aberration. In some embodiments, said impact score for a given nutrigenetic aberration of said one or more nutrigenetic aberrations is determined based on said scientific validity according to the rules and scores listed in Table 3. In some embodiments, determining said impact score for a given nutrigenetic aberration of said one or more nutrigenetic aberrations based on said clinical validity comprises determining said impact score based on one or more of biochemical impact of said given nutrigenetic aberration on clinical dysfunction or manifestation, existence of an intervention that modulates said biochemical impact, measurables and biomarkers of said given nutrigenetic aberration, and probability of benefit from said intervention. In some embodiments, said impact score for a given nutrigenetic aberration of said one or more nutrigenetic aberrations is determined based on said clinical validity according to the rules and scores listed in Table 4. In some embodiments, said one or more computer processors are individually or collectively programmed to further group said one or more impact scores that are part of the same metabolic pathway. In some embodiments, said one or more computer processors are individually or collectively programmed to further sum said groups of said one or more impact scores that are part of the same metabolic pathway. In some embodiments, said one or more computer processors are individually or collectively programmed to further express said summed impact scores as a percentage of the total pathway score to assign a pathway weighting to said metabolic pathway. In some embodiments, said one or more computer processors are individually or collectively programmed to further determine boundaries of biological states based on a maximum probable pathway score and a minimum probable pathway score from among a set of pathway scores. In some embodiments, said one or more computer processors are individually or collectively programmed to further determine boundaries of biological states using a supervised or unsupervised machine learning model. In some embodiments, said supervised machine learning model comprises a deep learning algorithm, a support vector machine (SVM), a neural network, or a Random Forest. In some embodiments, said unsupervised machine learning algorithm comprises a k-means clustering model or principal component analysis.

In another aspect, the present disclosure provides a non-transitory computer-readable medium comprising machine-executable code that, upon execution by one or more computer processors, implements a method for generating a nutrigenetic profile of a subject, the method comprising: (a) receiving genetic information of said subject, which genetic information comprises a plurality of nucleic acid sequences, wherein said genetic information is obtained by processing a biological sample obtained or derived from said subject using one or more nutrigenetic assays; (b) processing said genetic information to identify one or more nutrigenetic aberrations of said subject; (c) identifying one or more biological states corresponding to at least one of a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an adrenal pathway, an infection pathway, a circadian rhythm pathway, a mood pathway, a memory pathway, a carbohydrate metabolism pathway, a lipid metabolism pathway, a stress pathway, and a cardiovascular health pathway of said subject based at least in part on said one or more nutrigenetic aberrations identified in (b); and (d) outputting an electronic report indicative of said one or more biological states of said subject.

In some embodiments, said one or more nutrigenetic assays comprise one or more of: a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk (e.g., Alzheimer's), a migraine test, a thyroid test, an eczema test, and a cancer genetics assay. In some embodiments, said one or more nutrigenetic assays comprise two or more of: a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk (e.g., Alzheimer's), a migraine test, a thyroid test, an eczema test, and a cancer genetics assay. In some embodiments, said one or more nutrigenetic assays comprise three or more a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk (e.g., Alzheimer's), a migraine test, a thyroid test, an eczema test, and a cancer genetics assay.

In some embodiments, said one or more nutrigenetic aberrations comprise one or more of: single nucleotide polymorphisms (SNPs), copy number variants (CNVs), insertions or deletions (indels), fusions, and translocations. In some embodiments, said one or more nutrigenetic aberrations comprise at least about 100 distinct nutrigenetic aberrations. In some embodiments, wherein said one or more nutrigenetic aberrations comprise one or more of the nutrigenetic variants in Table 1 or Table 4.

In some embodiments, said cellular pathway comprises one or more of: detoxification, DNA damage, inflammation, methylation, and oxidative stress. In some embodiments, said functional systems pathway comprises one or more of: blood clotting, bone health, collagen and joints, brain health, glucose and insulin, sex hormone balance, vascular health, cognitive decline, memory loss, mood disorders and behavior, female sex hormone balance, male sex hormone balance, and histamine tolerance. In some embodiments, said energy pathway comprises one or more of: adipogenesis, appetite/satiety/intake, energy expenditure, exercise response, pro-inflammatory fat, weight gain and weight loss resistance, circadian rhythm, and starch metabolism. In some embodiments, said activity pathway comprises one or more of: training response (VO2max), endurance, injury, power, recovery, flexibility and strength. In some embodiments, said nutrients pathway comprises one or more of: caffeine, salt, vitamin D, fatty acids, vitamin A, vitamin B6, vitamin B12, folate, vitamin C, vitamin E, choline, iron overload, iron status, gluten, celiac, and cannaboid metabolism. In some embodiments, said cardiovascular health pathway comprises one or more of: blood clotting, blood pressure, vascular health, and lipid metabolism.

In some embodiments, (c) further comprises identifying said one or more biological states of said subject based at least in part on one or more of: a diagnosis of a disease or disorder, a prognosis of a disease or disorder, a risk of having a disease or disorder, a treatment history of a disease or disorder, a history of previous treatment for a disease or disorder, a history of prescribed medications, a history of prescribed medical devices, age, height, weight, sex, smoking status, and one or more symptoms of said subject. In some embodiments, said one or more symptoms comprise chronic fatigue, weight loss, nausea, insomnia, or a combination thereof.

In some embodiments, the method further comprises generating a nutrigenetic health regimen for said subject based at least in part on said identified one or more nutrigenetic aberrations. In some embodiments, said nutrigenetic health regimen comprises recommendations to prevent onset of a disease or disorder, delay onset of a disease or disorder, reverse a disease or disorder, or maintain a physiological or health state of said subject. In some embodiments, said nutrigenetic health regimen comprises recommendations related to one or more of: diet, exercise, sports training, supplements, functional tests, blood tests, brain management, behavior change, skin care, environmental exposure, stress management, and mental health.

In some embodiments, said electronic report is indicative of said nutrigenetic health regimen. In some embodiments, said electronic report is presented on a user interface (e.g., graphical user interface) of an electronic device of a user. In some embodiments, said user is said subject. In some embodiments, the method further comprises transmitting said electronic report to a remote user. In some embodiments, said remote user is a clinical practitioner or a nutrigenetics counselor. In some embodiments, the method further comprises storing said electronic report on a remote server.

In some embodiments, (c) comprises processing said identified one or more nutrigenetic aberrations using a trained algorithm to identify said one or more biological states. In some embodiments, said trained algorithm comprises a supervised machine learning algorithm. In some embodiments, said supervised machine learning algorithm comprises a deep learning algorithm, a support vector machine (SVM), a neural network, or a Random Forest. In some embodiments, said trained algorithm is configured to identify said one or more biological states at an accuracy of at least about 80% over 50 independent samples.

In some embodiments, said electronic report comprises one or more graphical depictions of said one or more biological states of said subject. In some embodiments, the method further comprises using said electronic report to provide said subject with a therapeutic intervention. In some embodiments, the method further comprises using said electronic report to treat said subject.

In another aspect, the present disclosure provides a method for providing nutrigenetic counseling for a subject, the method comprising: processing genetic information of said subject obtained using one or more nutrigenetic assays to identify one or more nutrigenetic aberrations of said subject; and generating a nutrigenetic regimen for said subject based at least in part on said identified one or more nutrigenetic aberrations, wherein said nutrigenetic regimen comprises instructions for maintaining or promoting a physiological or healthy state of said subject.

In some embodiments, the method further comprises using said nutrigenetic regimen to maintain or promote said physiological or healthy state of said subject.

In another aspect, the present disclosure provides a system for providing nutrigenetic counseling for a subject, comprising: a database configured to store genetic information of said subject obtained using one or more nutrigenetic assays; and one or more computer processors operatively coupled to said database, wherein said one or more computer processors are individually or collectively programmed to: (i) process said genetic information of said subject to identify one or more nutrigenetic aberrations of said subject; and (ii) generate a nutrigenetic regimen for said subject based at least in part on said identified one or more nutrigenetic aberrations, wherein said nutrigenetic regimen comprises instructions for maintaining or promoting a physiological or healthy state of said subject.

In another aspect, the present disclosure provides a non-transitory computer-readable medium comprising machine-executable code that, upon execution by one or more computer processors, implements a method for providing nutrigenetic counseling for a subject, the method comprising: processing genetic information of said subject obtained using one or more nutrigenetic assays to identify one or more nutrigenetic aberrations of said subject; and generating a nutrigenetic regimen for said subject based at least in part on said identified one or more nutrigenetic aberrations, wherein said nutrigenetic regimen comprises instructions for maintaining or promoting a physiological or healthy state of said subject.

In another aspect, the present disclosure provides a computer-implemented method for generating a nutrigenetic profile of a subject, the method comprising processing genetic information of said subject to identify one or more nutrigenetic aberrations, and using said one or more nutrigenetic aberrations to generate said nutrigenetic profile of said subject, which nutrigenetic profile includes one or more biological states corresponding to at least one of a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an adrenal pathway, an infection pathway, a circadian rhythm pathway, a mood pathway, a memory pathway, a carbohydrate metabolism pathway, a lipid metabolism pathway, a stress pathway, and a cardiovascular health pathway of said subject.

In another aspect, the present disclosure provides a system for generating a nutrigenetic profile of a subject, comprising: a database configured to store genetic information of said subject; and one or more computer processors operatively coupled to said database, wherein said one or more computer processors are individually or collectively programmed to: (i) process genetic information of said subject to identify one or more nutrigenetic aberrations, and (ii) use said one or more nutrigenetic aberrations to generate said nutrigenetic profile of said subject, which nutrigenetic profile includes one or more biological states corresponding to at least one of a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an adrenal pathway, an infection pathway, a circadian rhythm pathway, a mood pathway, a memory pathway, a carbohydrate metabolism pathway, a lipid metabolism pathway, a stress pathway, and a cardiovascular health pathway of said subject.

In another aspect, the present disclosure provides a non-transitory computer-readable medium comprising machine-executable code that, upon execution by one or more computer processors, implements a method for generating a nutrigenetic profile of a subject, the method comprising processing genetic information of said subject to identify one or more nutrigenetic aberrations, and using said one or more nutrigenetic aberrations to generate said nutrigenetic profile of said subject, which nutrigenetic profile includes one or more biological states corresponding to at least one of a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an adrenal pathway, an infection pathway, a circadian rhythm pathway, a mood pathway, a memory pathway, a carbohydrate metabolism pathway, a lipid metabolism pathway, a stress pathway, and a cardiovascular health pathway of said subject.

Another aspect of the present disclosure provides a non-transitory computer-readable medium comprising machine-executable code that, upon execution by one or more computer processors, implements any of the methods above or elsewhere herein.

Another aspect of the present disclosure provides a system comprising one or more computer processors and computer memory coupled thereto. The computer memory comprises machine-executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a flowchart for performing a nutrigenetic analysis for a subject, in accordance with disclosed embodiments.

FIGS. 2A and 2B show examples of a Gene Summary displayed to a user as part of a sample nutrigenetic report for a subject, in accordance with disclosed embodiments. Genes are placed into categories, such as “Beneficial,” “No Impact,” “Low,” “Medium,” “High,” and “Very High” to facilitate ease of understanding by the user.

FIG. 2C shows an example of Pathway-based Results displayed to a user as part of a sample nutrigenetic report for a subject, in accordance with disclosed embodiments. Pathways are placed into categories reflecting their level of impact, such as “Cellular,” “Systems,” “Energy,” “Activity,” and “Nutrients.” Further, the level of impact of the nutrigenetic variants on individual pathways can be listed, such as “Low,” “Medium,” “High,” and “Very High”.

FIGS. 2D and 2E show an example of Genes by Pathway displayed to a user as part of a sample nutrigenetic report for a subject, in accordance with disclosed embodiments. Genes are listed under tables that correspond to different categories of pathways (such as “Cellular,” “Systems,” “Energy,” “Activity,” and “Nutrients”) and columns within tables that correspond to different individual pathways, such as detoxification, DNA damage, inflammation, and methylation in the “Cellular” category; oxidative stress, blood clotting, bone/collagen/joints, brain health, glucose and insulin, sex hormone balance, and vascular health in the “Systems” category; adipogenesis, appetite/satiety/intake, energy expenditure, exercise response, pro-inflammatory fat, weight gain and weight loss resistance in the “Energy” category; endurance, injury, power, and recovery in the “Activity” category; and caffeine, salt, and vitamin D in the “Nutrients” category.

FIGS. 3A-3E show examples of overview descriptions of categories of pathways (such as “Cellular,” “Systems,” “Energy,” “Activity,” and “Nutrients”) displayed to a user as part of a sample nutrigenetic report for a subject, in accordance with disclosed embodiments.

FIG. 4 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a,” “an,” and “the” include plural references, unless the context clearly dictates otherwise. For example, the term “a biological sample” includes a plurality of biological samples, including mixtures thereof.

As used herein, the term “subject,” generally refers to an organism having testable or detectable genetic, nutrigenetic, or other health or other physiological parameter or information. A subject may be a person. The subject may be a vertebrate, such as, for example, a mammal. Non-limiting examples of mammals include humans, simians, farm animals, sport animals, and pets. The subject may be an organism, such as an animal, a plant, a fungus, an archaea, or a bacteria. The subject may be a human. The subject may be a non-human. The subject may have or be suspected of having a heath or physiological condition, such as a disease. In some examples, the subject is a patient. As an alternative, the subject may be asymptomatic with respect to the health or physiological condition (e.g., disease).

The term “biological sample,” as used herein, generally refers to a biological sample that may be obtained from a subject. Samples obtained from subjects may comprise a biological sample from a human, animal, plant, fungus, or bacteria. The sample may be obtained from a subject with a disease or disorder, from a subject that is suspected of having the disease or disorder, or from a subject that does not have or is not suspected of having the disease or disorder. The disease or disorder may be an infectious disease, an immune disorder or disease, a cancer, a genetic disease, a degenerative disease, a lifestyle disease, an injury, a rare disease, or an age related disease. The infectious disease may be caused by bacteria, viruses, fungi, and/or parasites. The sample may be taken before and/or after treatment of a subject with a disease or disorder. Samples may be taken during a treatment or a treatment regime. Multiple samples may be taken from a subject to monitor the effects of the treatment over time. The sample may be taken from a subject having or suspected of having a disease or disorder for which a definitive positive or negative diagnosis is not available via clinical tests.

A sample may be obtained from a subject suspected of having a disease or a disorder. The subject may be experiencing unexplained symptoms, such as fatigue, nausea, weight loss, aches and pains, weakness, or memory loss. The subject may have explained symptoms. The subject may be at risk of developing a disease or disorder due to factors such as familial history, age, environmental exposure, lifestyle risk factors, or presence of other known risk factors.

A sample may comprise a biological sample from a subject (e.g., human subject), such as saliva, cheek swab, blood, plasma, serum, cells, tissue (e.g., normal or tumor), urine, stool (feces), or derivatives or combinations thereof. The sample may be a tissue sample, such as a tumor sample. The sample may be a cell-free sample, such as a blood (e.g., whole blood), sweat, saliva or urine sample. The biological sample may be stored in a variety of storage conditions before processing, such as different temperatures (e.g., at room temperature, under refrigeration or freezer conditions, at 4° C., at −18° C., −20° C., or at −80° C.) or different preservatives (e.g., alcohol, formaldehyde, potassium dichromate, or EDTA).

As used herein, the term “nucleic acid” generally refers to a polymeric form of nucleotides of any length, either deoxyribonucleotides (dNTPs) or ribonucleotides (rNTPs), or analogs thereof. Nucleic acids may have any three dimensional structure, and may perform any function, known or unknown. Non-limiting examples of nucleic acids include deoxyribonucleic acid (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

The nucleic acid molecules may comprise deoxyribonucleic acid (DNA), ribonucleic acid (RNA) molecules, or a combination thereof. The DNA or RNA molecules may be extracted from the sample by a variety of methods, such as a FastDNA Kit protocol from MP Biomedicals. The extraction method may extract all DNA molecules from a sample. Alternatively, the extraction method may selectively extract a portion of DNA molecules from a sample, e.g., by targeting certain genes in the DNA molecules. Alternatively, extracted RNA molecules from a sample may be converted to DNA molecules by reverse transcription (RT). In some embodiments, after obtaining the sample, the sample may be processed to generate a plurality of genomic sequences. For example, processing the sample may comprise extracting a plurality of nucleic acid (DNA or RNA) molecules from the sample, and sequencing the plurality of nucleic acid (DNA or RNA) molecules to generate a plurality of nucleic acid (DNA or RNA) sequence reads.

The sequencing may be performed by any suitable sequencing method, such as massively parallel sequencing (MPS), paired-end sequencing, high-throughput sequencing, next-generation sequencing (NGS), shotgun sequencing, single-molecule sequencing (e.g., Pacific Biosciences of California), nanopore sequencing (e.g., Oxford Nanopore), semiconductor sequencing, pyrosequencing (e.g., 454 sequencing), sequencing-by-synthesis (SBS), sequencing-by-ligation, and sequencing-by-hybridization, or RNA-Seq (Illumina). Sequence identification may be performed using a genotyping approach such as an array. As an example, an array may be a microarray (e.g., Affymetrix or Illumina).

The sequencing may comprise nucleic acid amplification (e.g., of DNA or RNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., DNA) to a desired input quantity for subsequent sequencing or genotyping. In some cases, the PCR may be used for global amplification of nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing or genotyping. The PCR may comprise targeted amplification of one or more genomic loci, such as genomic loci corresponding to one or more diseases or disorders such as cancer markers (e.g., BRCA 1 and 2). The sequencing or genotyping may comprise use of simultaneous reverse transcription (RT) and polymerase chain reaction (PCR), such as a OneStep RT-PCR kit protocol provided by Qiagen, NEB, Thermo Fisher Scientific, or Bio-Rad.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase. For example, sequencing or genotyping of DNA molecules may be performed with or without amplification of DNA molecules.

DNA or RNA molecules may be tagged, e.g., with identifiable tags, to allow for multiplexing of a plurality of samples. Any number of DNA or RNA samples may be multiplexed. For example a multiplexed reaction may contain DNA or RNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial samples. For example, a plurality of samples may be tagged with sample barcodes such that each DNA or RNA molecule may be traced back to the sample (and the environment or the subject) from which the DNA or RNA molecule originated. Such tags may be attached to DNA or RNA molecules by ligation or by PCR amplification with primers.

After subjecting the nucleic acid molecules to sequencing, suitable bioinformatics processes may be performed on the sequence reads to generate the plurality of genomic sequences. For example, the sequence reads may be filtered for quality, trimmed to remove low quality, or aligned to one or more reference genomes (e.g., a human genome).

In some embodiments, after obtaining the biological sample, the biological sample may be processed to generate a proteome, metabolome, or any combination thereof. For example, processing the biological sample may comprise extracting a plurality of proteins from the biological sample, and analyzing the plurality of proteins to identify and/or quantify the plurality of proteins, thereby generating a proteome of the biological sample. As another example, processing the biological sample may comprise extracting a plurality of metabolites from the biological sample, and analyzing the plurality of metabolites to identify and/or quantify the plurality of metabolites, thereby generating a metabolome of the biological sample. The extraction method may extract all proteins and/or metabolites from a biological sample. Alternatively, the extraction method may selectively extract a portion of proteins and/or metabolites from a biological sample, e.g., by use of binding reagents such as probes or antibodies to target certain proteins and/or metabolites.

As used herein, a user can be an end-consumer, a company having at least one product that can analyze human nutrigenetic data to generate health-related recommendations and other information to an end-consumer; an entity that does not have any product but may also utilize the human nutrigenetic data for other purposes such as research; a subject from which the biological samples and/or nutrigenetic data are obtained; or a physician, nurse, nutrigenetic counselor, or other clinical provider.

The terms “nutrigenetic” and “nutrigenomic,” as used herein, generally refer to nutritional genetic or nutritional genomic information, such as relationships between a genome, nutrition, and health of a subject. For example, nutrigenetic analysis may be related to identifying or predicting heterogeneous or differential response of a subject to diet and nutrients based on analysis of nucleic acid sequences having gene variants, while nutrigenomics analysis may be related to the influence of diet and nutrients on the gene expression of a subject.

The term “nutrigenetic aberration,” as used herein, generally refers to a nutrition-related aberration in a genome of a subject, such as, for example, a nutrigenetic variant. A nutrigenetic aberration may be, for example, a genetic variation that has a relationship (e.g., a causative relationship or a highly correlated relationship, such as, for example, a correlation at an R²>0.8 or 0.9) with a nutrition and health of a subject, such as a single nucleotide polymorphisms (SNP), copy number variation (CNV), insertion or deletion (indel), fusion, or translocation. A nutrigenetic variant may be, for example, a genetic variant associated with differential response to nutrients (e.g., differential gene expression or DNA methylation).

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

A large number of diseases or disorders may arise at least in part because of a genetic or nutrigenetic basis. Thus, analysis of genetic, nutrigenetic, or other types of data of subjects may provide valuable insights into disease causes and risks as well as lifestyle recommendations for a subject to manage his or her own health. However, nutrigenetic assays may generate complex datasets that are difficult to interpret and understand by an end user, such as a subject or patient. In particular, nutrigenomics or nutrigenetic data generated from one or more genetic assays may need to be efficiently collected, analyzed, and interpreted to understand how unique genetic instructions can determine the way a subject's body responds to dietary and environmental factors such as food, exercise, stress, and toxins. Thus, there is a need for accurate and effective reporting of nutrigenomics or nutrigenetic data that is comprehensive, clinically tested, and easy to understand, as well as translation of such nutrigenomics or nutrigenetic data into a practical plan of actionable, personalized recommendations for how a subject can positively impact his or her own health. Such accurate and effective reporting of nutrigenomics or nutrigenetic data may represent significant improvements in at least the technical fields of nutrigenomics and/or nutrigenetic data reporting, nutrigenomics and/or nutrigenetic data analysis, nutrigenomics and/or nutrigenetic counseling of subjects (e.g., patients), nutrigenomics and/or nutrigenetic data management, and clinical translation of nutrigenomics and/or nutrigenetic reports.

The present disclosure provides methods and systems for nutrigenomics and nutrigenetic analysis, including generating a nutrigenetic profile of a subject and/or reporting of the nutrigenetic profile or nutrigenetic data to a user. Although analysis of human genetic data, such as nutrigenomics data and/or nutrigenetic data, may produce significant insights toward advancing understanding of diseases and disorders, there can be concerns about accurate and effective reporting of nutrigenetic data that is comprehensive, clinically tested, and easy to understand. In addition, such nutrigenetic data may need to be translated into a practical plan of actionable, personalized recommendations for how a subject can positively impact his or her own health.

### Nutrigenetic Analysis

In an aspect, the present disclosure provides a computer-implemented method for generating a nutrigenetic profile of a subject. The method may comprise receiving genetic information of the subject comprising a plurality of nucleic acid sequences, wherein the genetic information is obtained by processing a biological sample obtained or derived from the subject using one or more nutrigenetic assays. Next, the genetic information may be processed to identify one or more nutrigenetic variants of the subject. One or more biological states may then be identified corresponding to at least one of (i) a metabolic pathway, (ii) a cellular pathway, (iii) a functional systems pathway, (iv) an energy pathway, (v) an activity pathway, (vi) a nutrients pathway, (vii) a skin pathway, (viii) an immune pathway, (ix) a gut pathway, (x) a thyroid pathway, (xi) a mitochondria health pathway, (xii) an infection pathway, (xiii) a circadian rhythm pathway, (xiv) a mood pathway, (xv) a memory pathway, (xvi) a carbohydrate metabolism pathway, (xvii) a lipid metabolism pathway, (xviii) a stress pathway, and (xix) an adrenal pathway of the subject based at least in part on the identified one or more nutrigenetic variants. An electronic report indicative of the one or more biological states of the subject may then be outputted.

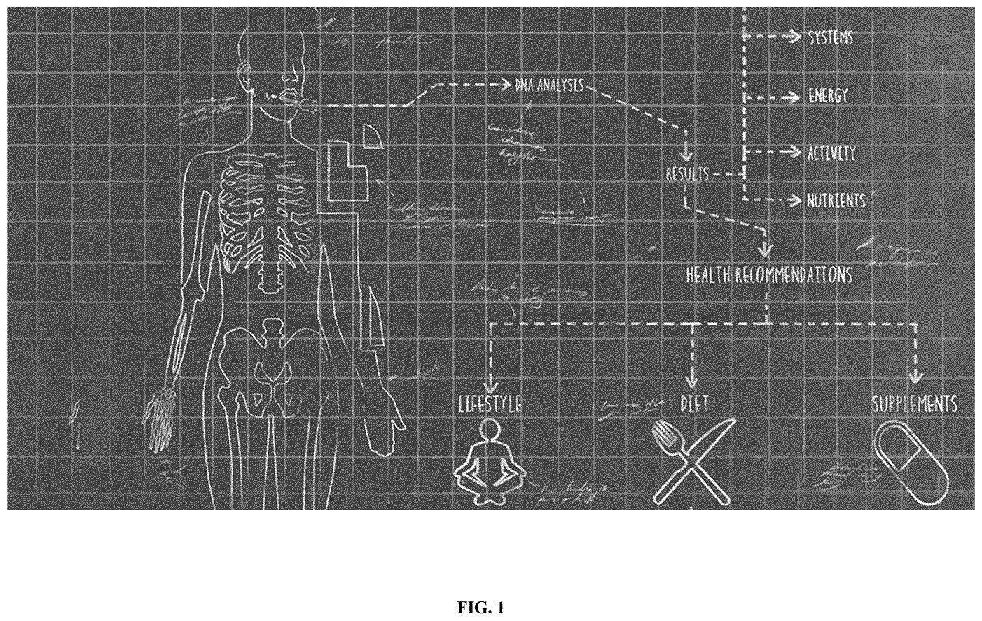

FIG. 1 shows an example of a flowchart for performing a nutrigenetic analysis for a subject, in accordance with disclosed embodiments. Such analysis may include a method 100 for generating a nutrigenetic profile and/or report of the subject. First, in operation 102, analysis (e.g., DNA analysis) may be performed on a biological sample obtained or derived from a subject to generate a set of data or results 104. For example, the analysis may be performed using one or more nutrigenetic assays. The data or results may comprise one or more of: genomic data (e.g., DNA sequences), transcriptomic data (e.g., RNA sequences), proteomic data (e.g., identification and/or quantification of proteins in the biological sample), metabolomic data (e.g., identification and/or quantification of metabolites in the biological sample), or a combination thereof. Next, the data or results may be analyzed to identify one or more nutrigenetic aberrations (e.g., nutrigenetic variants) of the subject, which may then be categorized and/or displayed based on different pathways or categories of pathways (e.g., pathways corresponding to systems 106, pathways corresponding to energy 108, pathways corresponding to activity 110, and/or pathways corresponding to nutrients 112). Next, one or more biological states (e.g., corresponding to a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an infection pathway, a circadian rhythm pathway, a mood pathway, a memory pathway, a carbohydrate metabolism pathway, an adrenal pathway, a lipid metabolism pathway, and/or a stress pathway) of the subject may be identified based at least in part on the identified one or more nutrigenetic variants. Next, in operation 114, a set of health recommendations may then be generated based at least in part on the pathway-based analysis of the data or results (e.g., the identified biological states). For example, the set of health recommendations may comprise recommendations related to the subject's lifestyle 116, diet 118, supplements 120, exercise, sports training, functional tests, blood tests, brain management, behavioral change, environmental exposure, skin care, stress management, or a combination thereof. An electronic report may be generated and outputted which is indicative of the nutrigenetic variants of the subject (which may then be categorized and/or displayed based on different pathways or categories of pathways), the biological states of the subject, the set of health recommendations for the subject, or a combination thereof. The electronic report may contain a visual representation of the results (e.g., nutrigenetic variants and pathway-based analysis) for ease of understanding by a user.

The nutrigenetic assays may be used to assay the biological sample to generate genomic or genetic information or data (e.g., related to nutrigenetic aberrations or variants). In some embodiments, the nutrigenetic assays comprise one or more of: a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk (e.g., Alzheimer's), a migraine test, a thyroid test, an eczema test, and a cancer genetics assay. For example, at least 1, 2, 3, 4, 5, 6, 7, 8, 9, 10, or more than 10 distinct nutrigenetic assays may be used to assay the biological sample to generate genetic information or data. The nutrigenetic aberrations can comprise one or more of: single nucleotide polymorphisms (SNPs), copy number variants (CNVs), insertions or deletions (indels), fusions, and translocations. The one or more nutrigenetic aberrations may comprise at least about 5, at least about 10, at least about 15, at least about 20, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 350, at least about 400, at least about 450, at least about 500, or more than about 500 distinct nutrigenetic aberrations. In some embodiments, the one or more nutrigenetic aberrations comprise one or more of the nutrigenetic variants in Table 1 or Table 4.

In some embodiments, biological states of the subject are identified based at least in part on the one or more nutrigenetic aberrations. Biological states may correspond to, for example, a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an infection pathway, a circadian rhythm pathway, a mood pathway, a memory pathway, a carbohydrate metabolism pathway, an adrenal pathway, a lipid metabolism pathway, a stress pathway, or a combination thereof. In some embodiments, the cellular pathway comprises one or more of: detoxification, DNA damage, inflammation, methylation, and oxidative stress. In some embodiments, the functional systems pathway comprises one or more of: blood clotting, bone health, collagen and joints, brain health, glucose and insulin, sex hormone balance, vascular health, cognitive decline, memory loss, mood disorders and behavior, female sex hormone balance, male sex hormone balance, and histamine tolerance. In some embodiments, the energy pathway comprises one or more of: adipogenesis, appetite/satiety/intake, energy expenditure, exercise response, pro-inflammatory fat, weight gain and weight loss resistance, circadian rhythm, and starch metabolism. In some embodiments, the activity pathway comprises one or more of: training response (VO2max), endurance, injury, power, recovery, flexibility and strength. In some embodiments, the nutrients pathway comprises one or more of: caffeine, salt, vitamin D, fatty acids, vitamin A, vitamin B6, vitamin B12, folate, vitamin C, vitamin E, choline, iron overload, iron status, gluten, celiac, and cannaboid metabolism. In some embodiments, the cardiovascular health pathway comprises one or more of: blood clotting, blood pressure, vascular health, and lipid metabolism.

In some embodiments, the one or more biological states of the subject are identified based at least in part on additional clinical information of the subject, such as one or more of: a diagnosis of a disease or disorder, a prognosis of a disease or disorder, a risk of having a disease or disorder, a treatment history of a disease or disorder, a history of previous treatment for a disease or disorder, a history of prescribed medications, a history of prescribed medical devices, age, height, weight, sex, smoking status, and one or more symptoms of the subject. In some embodiments, the one or more symptoms comprise chronic fatigue, weight loss, nausea, insomnia, or a combination thereof.

After the nutrigenetic aberrations of the subject are identified, the method may further comprise generating a nutrigenetic health regimen for the subject based at least in part on the identified one or more nutrigenetic aberrations (e.g., nutrigenetic variants). For example, the nutrigenetic health regimen may comprise recommendations to prevent onset of a disease or disorder, delay onset of a disease or disorder, reverse a disease or disorder, maintain a physiological or health state of the subject, or a combination thereof. In some embodiments, the nutrigenetic health regimen comprises recommendations related to one or more of: diet, exercise, sports training, supplements, functional tests, blood tests, brain management, behavior change, skin care, environmental exposure, stress management, and mental health.

The nutrigenetic health regimen may be generated based at least in part on additional clinical information of the subject, such as one or more of: a diagnosis of a disease or disorder, a prognosis of a disease or disorder, a risk of having a disease or disorder, a treatment history of a disease or disorder, a history of previous treatment for a disease or disorder, a history of prescribed medications, a history of prescribed medical devices, age, height, weight, sex, smoking status, and one or more symptoms of the subject. In some embodiments, the one or more symptoms comprise chronic fatigue, weight loss, nausea, insomnia, or a combination thereof.

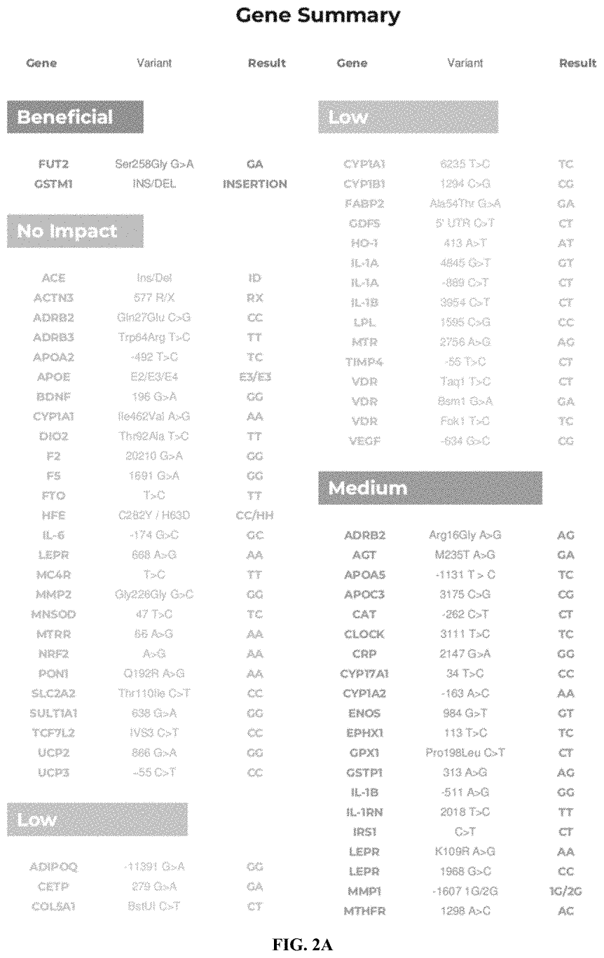

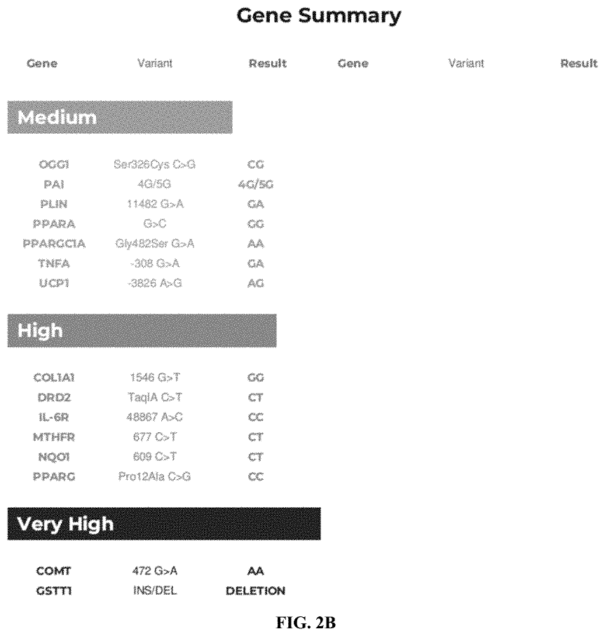

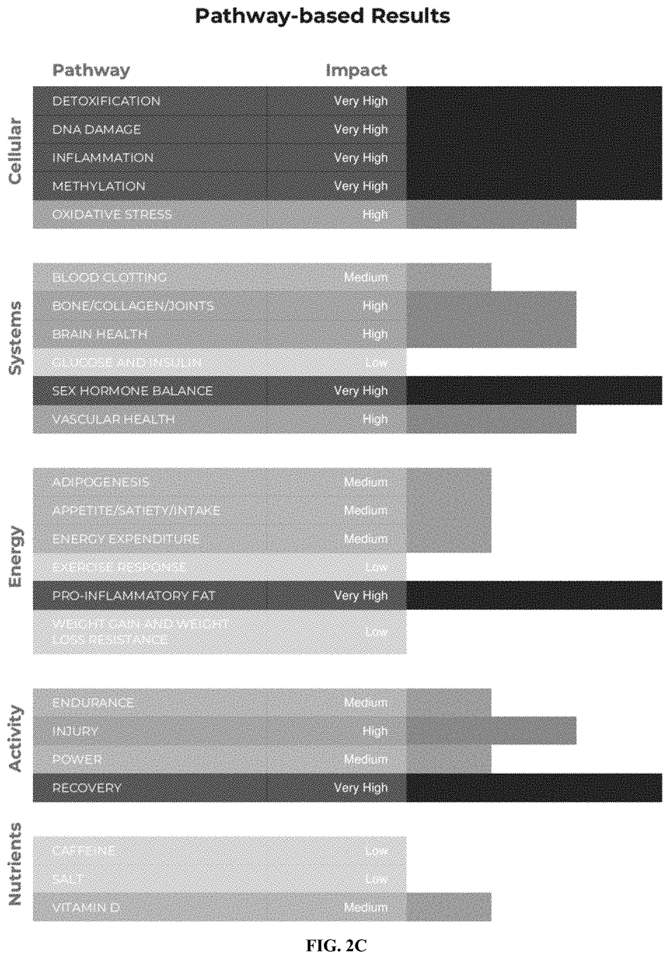

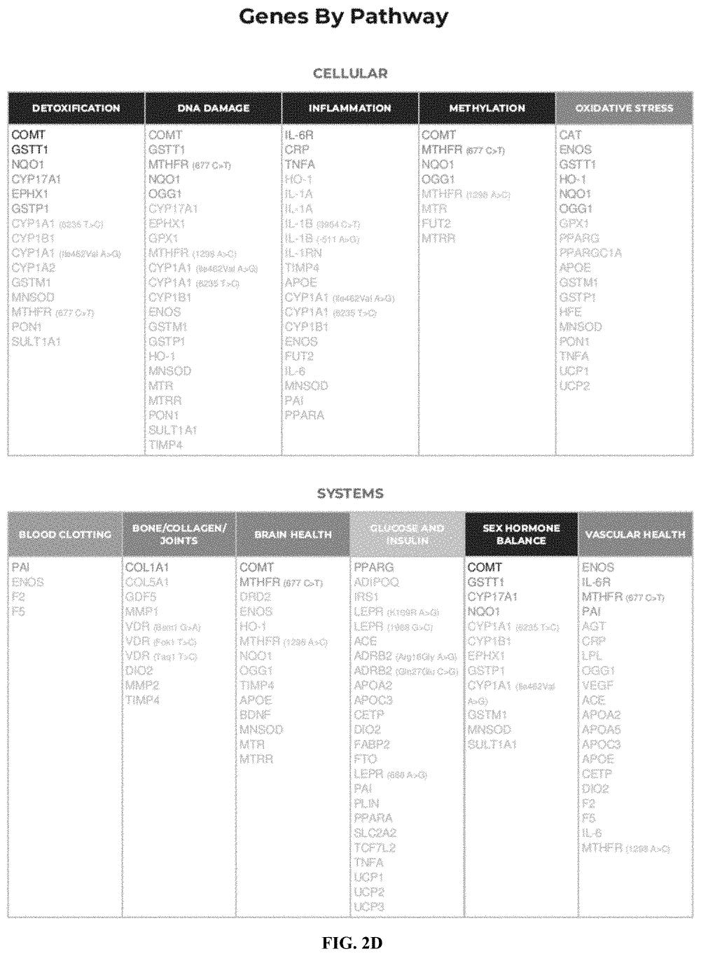

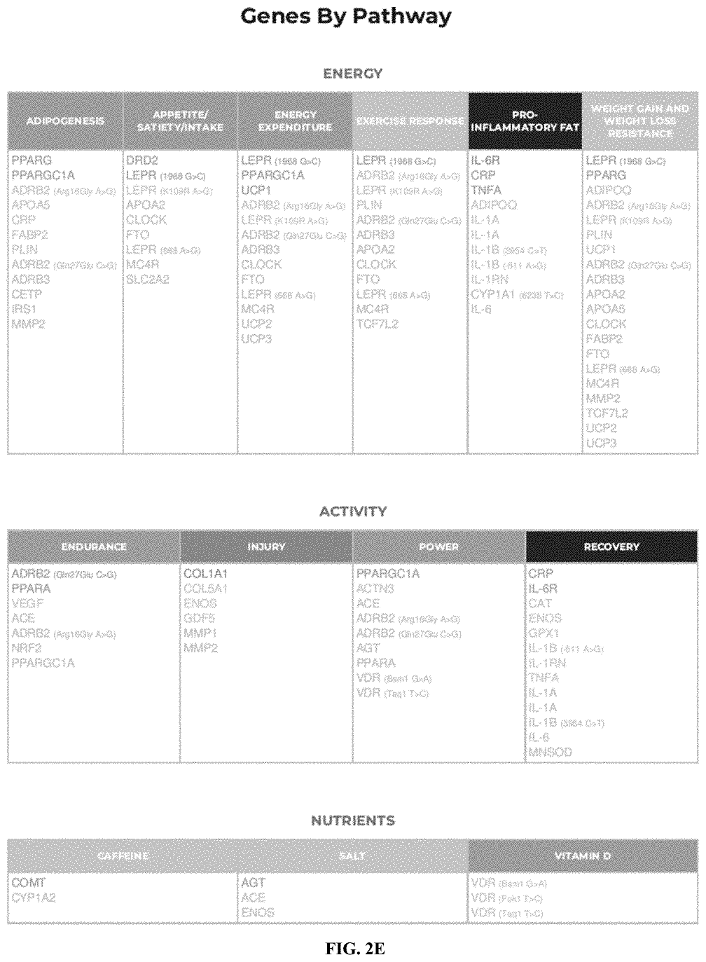

FIGS. 2A and 2B show examples of a Gene Summary displayed to a user as part of a sample nutrigenetic report for a subject, in accordance with disclosed embodiments. Genes are placed into categories reflecting their level of impact, such as “Beneficial,” “No Impact,” “Low,” “Medium,” “High,” and “Very High” to facilitate ease of understanding by the user. For example, genes in the “Beneficial” category may include genes having one or more nutrigenetic variants that confer a beneficial impact to the subject through one or more pathways. As another example, genes in the “No Impact” category may include genes having one or more nutrigenetic variants that confer no significantly beneficial or detrimental impact to the subject through one or more pathways. As another example, genes in the “Low,” “Medium,” “High,” and “Very High” categories may include genes having one or more nutrigenetic variants that confer a detrimental impact to the subject through one or more pathways. For ease of visual understanding, each of the categories may be displayed in a different color code according to its impact. For each of the genes listed, a nutrigenetic variant of the gene and/or a result of the nutrigenetic variant can be listed (e.g., “Ins/Del” to denote an insertion or deletion (indel), and “C>G” to denote a substitution of a “C” residue with a “G” residue at a given position).

FIG. 2C shows an example of Pathway-based Results displayed to a user as part of a sample nutrigenetic report for a subject, in accordance with disclosed embodiments. Pathways are placed into categories, such as “Cellular,” “Systems,” “Energy,” “Activity,” and “Nutrients.” Such categorical indications can inform a user about different types of pathways that are impacted by the nutrigenetic variants identified in the subject, thereby facilitating an improved understanding by the user of the clinical significance of the results displayed in the nutrigenetic report. Further, the level of impact of the nutrigenetic variants on individual pathways can be listed, such as “Low,” “Medium,” “High,” and “Very High”. For ease of visual understanding, each of the pathways may be displayed in a different color code according to its impact.

FIGS. 2D and 2E show an example of Genes by Pathway displayed to a user as part of a sample nutrigenetic report for a subject, in accordance with disclosed embodiments. Genes are listed under tables that correspond to different categories of pathways (such as “Cellular,” “Systems,” “Energy,” “Activity,” and “Nutrients”) and columns within tables that correspond to different individual pathways, such as detoxification, DNA damage, inflammation, and methylation in the “Cellular” category; oxidative stress, blood clotting, bone/collagen/joints, brain health, glucose and insulin, sex hormone balance, and vascular health in the “Systems” category; adipogenesis, appetite/satiety/intake, energy expenditure, exercise response, pro-inflammatory fat, weight gain and weight loss resistance in the “Energy” category; endurance, injury, power, and recovery in the “Activity” category; and caffeine, salt, and vitamin D in the “Nutrients” category. For ease of visual understanding, each of the genes may be displayed in a different color code according to its pathway category, pathway, and/or impact.

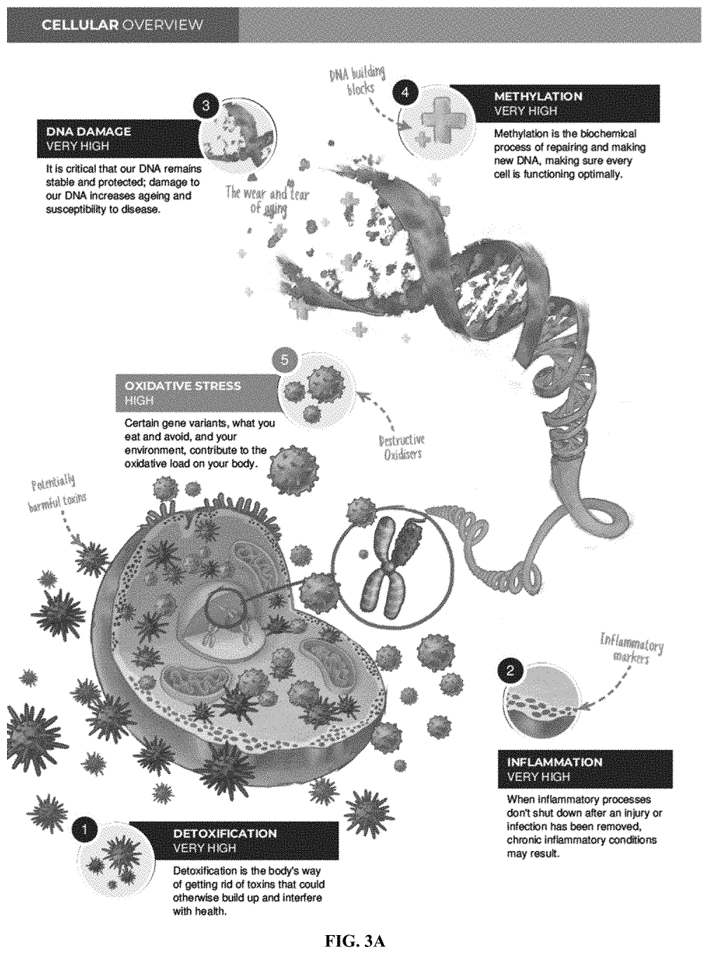

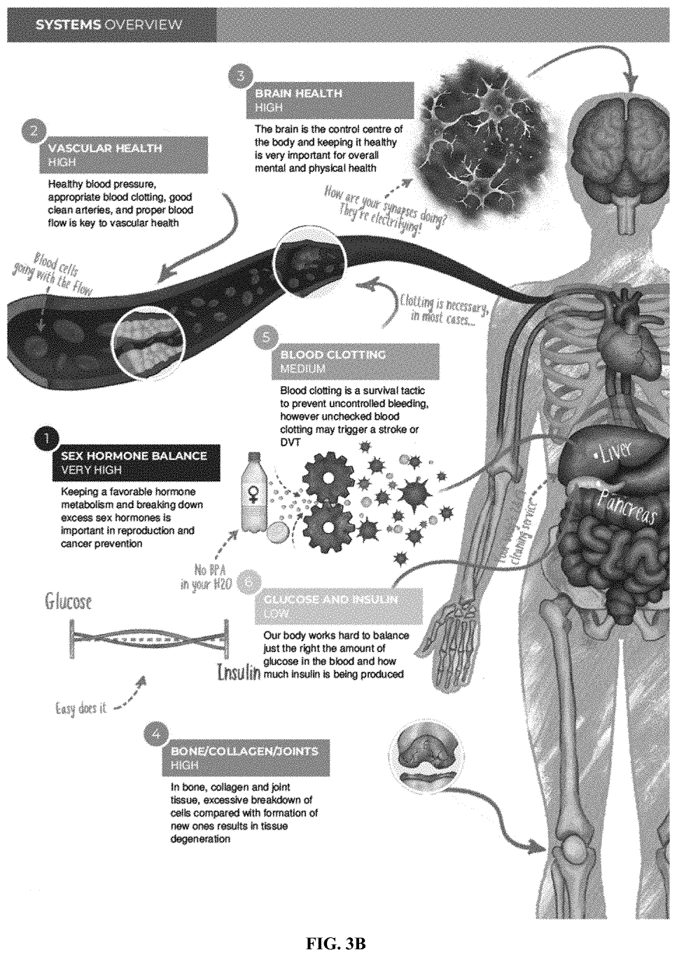

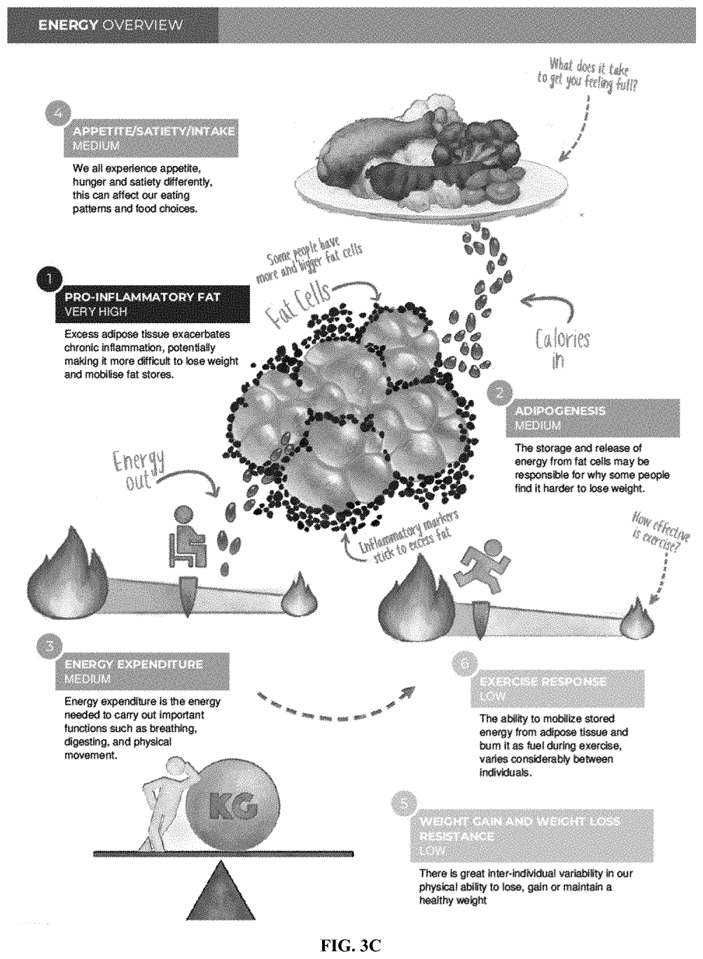

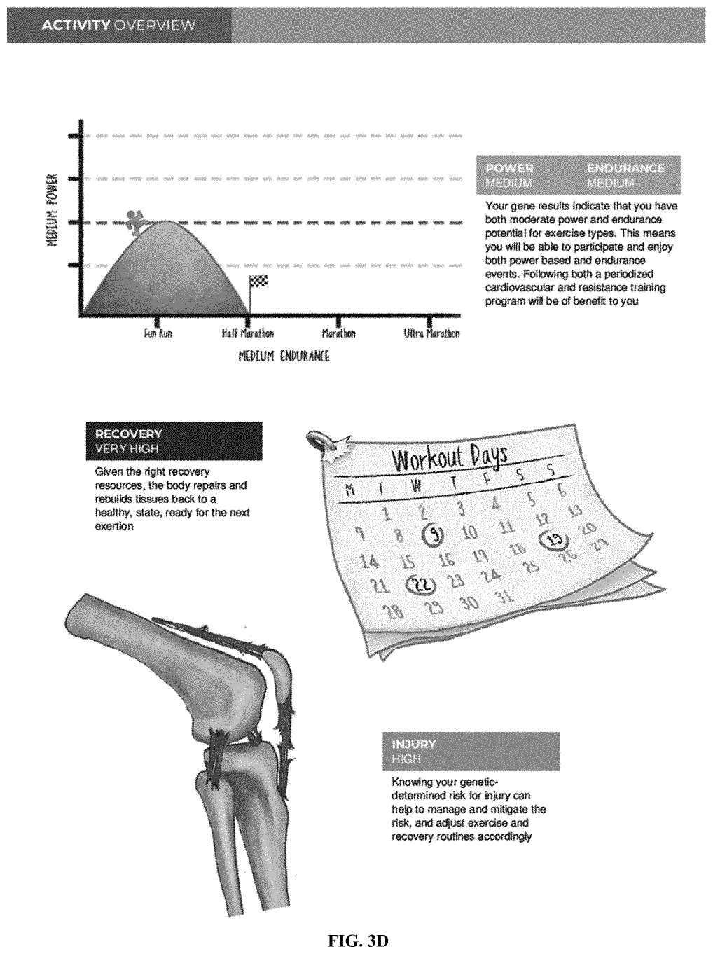

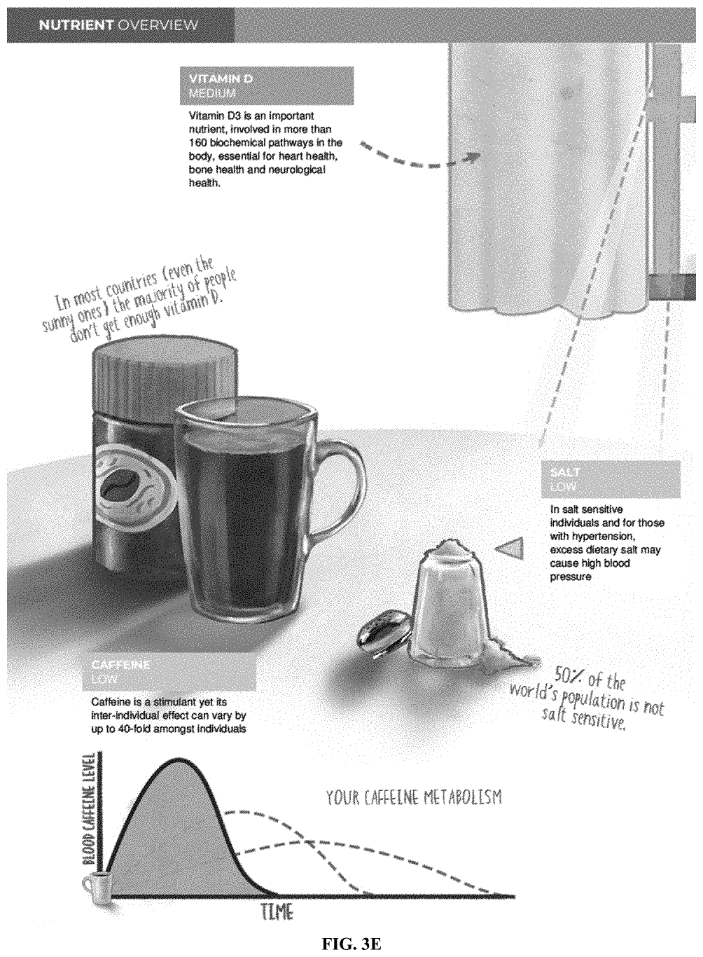

FIGS. 3A-3E show examples of overview descriptions of categories of pathways (such as “Cellular,” “Systems,” “Energy,” “Activity,” and “Nutrients”) displayed to a user as part of a sample nutrigenetic report for a subject, in accordance with disclosed embodiments. Such categorical descriptions can inform a user about possible nutrigenetic impacts on different pathways based on the subject's nutrigenetic profile, thereby facilitating an improved understanding by the user of the clinical significance of the results displayed in the nutrigenetic report. The impact levels ascribed to these pathways are specific to each subject's set of genetic results and will therefore be different for each individual nutrigenetic report.

FIG. 3A is an overview description of the “Cellular” category of pathways. First, the detoxification pathway, which has a very high impact, is related to detoxification, which is the body's way of getting rid of toxins that could otherwise build up and interfere with health. Second, the inflammation pathway, which has a very high impact, is related to chronic inflammatory conditions that may result when inflammatory processes are chronic and sustained rather than those experienced after an injury or infection. Third, the DNA damage pathway, which has a very high impact, is related to increased ageing and susceptibility to disease. Fourth, the methylation pathway, which has a very high impact, is related to methylation, the biochemical process of repairing and making new DNA to ensure every cell is functioning optimally. Fifth, the oxidative stress pathway, which has a high impact, is related to a subject's diet, lifestyle, and environmental exposures, which contribute to the oxidative load on the subject's body.

FIG. 3B is an overview description of the “Systems” category of pathways. First, the sex hormone balance pathway, which has a very high impact, is related to the importance of keeping a favorable hormone metabolism and breaking down excess endogenous and exogenous sex hormones toward reproduction and cancer prevention. Second, the vascular health pathway, which has a high impact, is related to maintaining healthy blood pressure, appropriate blood clotting, good clean arteries, and proper blood flow. Third, the brain health pathway, which has a high impact, is related to keeping the brain healthy for overall optimal cognition. Fourth, the bone/collagen/joints pathway, which has a high impact, is related to tissue modeling and degeneration, which results from excessive breakdown of cells compared with formation of new ones in bone, collagen, and joint tissue. Fifth, the blood clotting pathway, which has a medium impact, is related to blood clotting, which is a survival tactic to prevent uncontrolled bleeding, but if unchecked may trigger a stroke or deep vein thrombosis (DVT). Sixth, the glucose and insulin pathway, which has a low impact, is related to the body's regulation of the right amount of glucose in the blood and how much insulin is being produced.

FIG. 3C is an overview description of the “Energy” category of pathways. First, the pro-inflammatory fat pathway, which has a very high impact, is related to excess adipose tissue, which exacerbates chronic inflammation, potentially making it more difficult to lose weight and mobilize fat stores. Second, the adipogenesis pathway, which has a medium impact, is related to the storage and release of energy from fat cells, which may be responsible for why some people find it harder to lose weight and mobilize fat stores. Third, the energy expenditure pathway, which has a medium impact, is related to the energy needed to carry out important functions, such as breathing, digesting, and physical movement. Fourth, the appetite/satiety/intake pathway, which has a medium impact, is related to different experiences of appetite, hunger, and satiety, which can affect a subject's eating patterns and food choices. Fifth, the weight gain and weight loss resistance pathway, which has a low impact, is related to inter-individual variability in a subject's physical ability to lose, gain, or maintain a healthy weight. Sixth, the exercise response pathway, which has a low impact, is related to the ability to mobilize stored energy from adipose tissue and burn it as fuel during exercise, which varies considerably between individuals.

FIG. 3D is an overview description of the “Activity” category of pathways. First, the power and endurance pathways, which have a medium impact, can indicate that a subject has both moderate power and endurance potential for exercise types, which means the subject will be able to participate and enjoy both power based and endurance events and that following both a periodized cardiovascular and resistance training program will be of benefit to the subject. Second, the recovery pathway, which has a very high impact, is related to the body's ability to repair and rebuild tissues back to a healthy state after an exercise bout, ready for the next exertion. Third, the injury pathway, which has a high impact, is related to a subject's genetic-determined risk for collagen-based injuries, which can be used to help manage and mitigate the risk, and adjust exercise and recovery routines accordingly.

FIG. 3E is an overview description of the “Nutrients” category of pathways. First, the vitamin D pathway, which has a medium impact, is related to the effective metabolism of vitamin D, which is an important nutrient involved in more than 160 biochemical pathways in the body, and is essential for heart health, bone health, and neurological health. Second, the salt pathway, which has a low impact, is related to an individual's response to dietary salt, as salt-sensitive individuals are more prone to hypertension. Third, the caffeine pathway, which has a low impact, is related to caffeine's stimulant effect on a subject, which can vary by up to 40-fold amongst individuals.

### User Portals and Platforms

In another aspect, the present disclosure provides a system for generating a nutrigenetic profile of a subject. The system may comprise a database configured to store genetic information of the subject, which genetic information comprises a plurality of nucleic acid sequences, and one or more computer processors operatively coupled to the database. The one or more computer processors may be individually or collectively programmed to: (i) process the genetic information to identify one or more nutrigenetic aberrations of the subject; (ii) identify one or more biological states corresponding to at least one of a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an infection pathway, a circadian rhythm pathway, a mood pathway, a memory pathway, a carbohydrate metabolism pathway, a lipid metabolism pathway, a stress pathway, and a cardiovascular health pathway of the subject based at least in part on the one or more nutrigenetic aberrations identified in (ii); and (iii) electronically output a report indicative of the one or more biological states of the subject. In some embodiments, the genetic information is obtained by processing a biological sample obtained or derived from the subject using one or more nutrigenetic assays.

The system may generate the nutrigenetic profile of the subject, facilitate data exchange of the nutrigenetic profile among end users (e.g., using a network such as a cloud network), store the nutrigenetic profile in a database (e.g., a cloud network), and/or display an electronic report comprising the nutrigenetic profile to an end user.

The system may facilitate data exchange of the nutrigenetic profile among end users (e.g., using a network such as a cloud network) and/or store the nutrigenetic profile in a database (e.g., a cloud network). The system may comprise a network interface that is in network communication with digital computers of different users. The network interface may include a portal or a platform, such as a user portal (e.g., for an end user to view nutrigenetic profiles) or a clinician portal (e.g., for a clinician to view or annotate nutrigenetic profiles). In some embodiments, a cloud-based method or system can be provided to a user for facilitating nutrigenetic data exchange. The user can use a web-application to log in and access his nutrigenetic data over a cloud-based computer system in the application, wherein the nutrigenetic data is generated from processing at least one biological sample of the user.

The systems and methods provided herein can include a user portal and/or a user platform that is configured to perform nutrigenetic analysis, display nutrigenetic profiles and reports to a user and/or control access to nutrigenetics profiles, reports, and/or data. The user portal and/or user platform may include a server that includes a digital processing device or a processor that can execute machine code, such as a computer program or algorithm, to enable one or more method steps or operations, as disclosed herein. Such computer programs or algorithms can be run automatically or on-demand based on one or more inputs from the users. The user portal and/or a user platform may allow users to connect with each other via the portal or platform, such as for nutrigenetic data exchange, thereby forming a network of connected users. Such data exchange can be secure and/or cloud-based. The users may each have an account for accessing the network and utilizing the functions associated with nutrigenetic data exchange securely and conveniently. The portal and/or platform may include a user interface, e.g., graphical user interface (GUI). The portal and/or platform may include a web application or mobile application. The portal and/or platform may include a digital display to display information to the user and/or an input device that can interact with the user to accept input from the user.

In some embodiments, the electronic report comprising nutrigenetic data or health regimens are presented on a user interface, such as a graphical user interface (GUI), of an electronic device of a user (e.g., the subject). The electronic report may be transmitted to a remote user (e.g., a clinical practitioner or a nutrigenetics counselor). Further, the electronic report can be stored on a remote server (e.g., a cloud-based server).

### Classifiers

After processing the biological sample from the subject, the nutrigenetic profiling method may comprise processing a set of identified nutrigenetic aberrations (e.g., nutrigenetic variants) of a subject using a trained algorithm (e.g., a classifier) to identify one or more biological states of the subject. The classifier may be used to classify the biological sample as corresponding to one or more biological states of the subject. The classifier may comprise a supervised machine learning algorithm or an unsupervised machine learning algorithm. The classifier may comprise a classification and regression tree (CART) algorithm. The classifier may comprise, for example, a support vector machine (SVM), a linear regression, a logistic regression, a nonlinear regression, a neural network, a Random Forest, a deep learning algorithm, a naive Bayes classifier. The classifier may comprise an unsupervised machine learning algorithm, e.g., clustering analysis (e.g., k-means clustering, hierarchical clustering, mixture models, DBSCAN, OPTICS algorithm), principal component analysis, independent component analysis, non-negative matrix factorization, singular value decomposition, anomaly detection (e.g., local outlier factor), neural network (e.g., autoencoder, deep belief network, Hebbian learning, generative adversarial network, self-organizing map), expectation-maximization algorithm, and method of moments.

The classifier may be configured to accept a plurality of input variables and to produce one or more output values based on the plurality of input variables. The plurality of input variables may comprise data indicative of a set of identified nutrigenetic aberrations (e.g., nutrigenetic variants). For example, an input variable may comprise a set of identified nutrigenetic variants or alleles, and/or a number of sequences corresponding to or aligning to each of the set of identified nutrigenetic variants or alleles.

The classifier may have one or more possible output values, each comprising one of a fixed number of possible values (e.g., a linear classifier, a logistic regression classifier, etc.) indicating a classification of the biological sample into a biological state (e.g., level of impact of an allele on a pathway). The classifier may comprise a binary classifier, such that each of the one or more output values comprises one of two values (e.g., {0, 1}, {positive, negative}, or {impact, no impact}, {diseased, non-diseased}) indicating a classification of the biological sample into a biological state (e.g., level of impact). The classifier may be another type of classifier, such that each of the one or more output values comprises one of more than two values (e.g., {0, 1, 2}, {positive, negative, or indeterminate}, {beneficial impact, low impact, medium impact, high impact, and/or very high impact}, or {diseased, non-diseased, or indeterminate}) indicating a classification of the biological sample into a biological state (e.g., level of impact). The output values may comprise descriptive labels, numerical values, or a combination thereof. Some of the output values may comprise descriptive labels. Such descriptive labels may provide an identification or indication of the biological state (e.g., level of impact) of the subject, and may comprise, for example, beneficial impact, low impact, medium impact, high impact, and/or very high impact . Such descriptive labels may provide an identification of a recommendation for the subject's biological state (e.g., level of impact), and may comprise, for example, a therapeutic intervention, a duration of the therapeutic intervention, and/or a recommendation related to diet, exercise, sports training, supplements, functional tests, blood tests, brain management, behavior change, skin care, environmental exposure, stress management, and/or mental health. Such descriptive labels may provide an identification of secondary clinical tests that may be appropriate to perform on the subject, and may comprise, for example, a biopsy, a blood test, a functional test, a computed tomography (CT) scan, a magnetic resonance imaging (MRI) scan, an ultrasound scan, a chest X-ray, a positron emission tomography (PET) scan, or a PET-CT scan. Such descriptive labels may provide a prognosis of the disease state of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}. Such integer output values may comprise, for example, {0, 1, 2}. Such continuous output values may comprise, for example, a probability value of at least 0 and no more than 1 (e.g., of the classification of the biological sample into a biological state). Such continuous output values may comprise, for example, an un-normalized probability value of at least 0. Such continuous output values may comprise, for example, an un-normalized probability value of at least 0. Such continuous output values may comprise, for example, an indication of an expected duration of an intervention. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of being recommended an intervention as a result of the impact on a pathway or nutrigenetic variant. For example, a binary classification of samples may assign an output value of “negative” or 0 if the sample indicates that the subject has less than a 50% probability of being recommended an intervention as a result of the impact on a pathway or nutrigenetic variant. In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, and about 99%.

As another example, a classification of samples may assign an output value of “positive” or 1 if the sample indicates that the subject has a probability of being recommended an intervention of at least 50%, at least about 55%, at least about 60%, at least about 65%, at least about 70%, at least about 75%, at least about 80%, at least about 85%, at least about 90%, at least about 95%, at least about 98%, or at least about 99%. The classification of samples may assign an output value of “positive” or 1 if the sample indicates that the subject has a probability of being recommended an intervention of more than 50%, more than 55%, more than 60%, more than 65%, more than 70%, more than 75%, more than 80%, more than 85%, more than 90%, more than 95%, more than 98%, or more than 99%. The classification of samples may assign an output value of “negative impact” or 0 if the sample indicates that the subject has a probability of being recommended an intervention of less than 50%, less than 45%, less than 40%, less than 35%, less than 30%, less than 25%, less than 20%, less than 10%, less than 5%, less than 2%, or less than 1%. The classification of samples may assign an output value of “negative” or 0 if the sample indicates that the subject has a probability of being recommended an intervention of no more than 50%, no more than 45%, no more than 40%, no more than 35%, no more than 30%, no more than 25%, no more than 20%, no more than 10%, no more than 5%, no more than 2%, or no more than 1%. The classification of samples may assign an output value of “indeterminate” or 2 if the sample has not been classified as “positive,” “negative,” 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The classifier may be trained with a plurality of independent training samples. Each of the independent training samples may comprise a biological sample from a subject, associated data obtained by processing the biological sample (as described elsewhere herein), and one or more known output values corresponding to biological states of the biological sample. Independent training samples may comprise biological samples and associated data and outputs obtained from a plurality of different subjects. Independent training samples may comprise biological samples and associated data and outputs obtained at a plurality of different time points from the same subject. Independent training samples may be associated with presence of a biological state (e.g., training samples comprising biological samples and associated data and outputs obtained from a plurality of subjects known to have the biological state). Independent training samples may be associated with absence of a biological state (e.g., training samples comprising biological samples and associated data and outputs obtained from a plurality of subjects who are known to not have the biological state).

The classifier may be trained with at least about 50, at least about 100, at least about 150, at least about 200, at least about 250, at least about 300, at least about 350, at least about 400, at least about 450, or at least about 500 independent training samples. The independent training samples may comprise samples associated with presence of the biological state and/or samples associated with absence of the biological state. The classifier may be trained with no more than 500, no more than 450, no more than 400, no more than 350, no more than 300, no more than 250, no more than 200, no more than 150, no more than 100, or no more than 50 independent training samples associated with presence of the biological state. In some embodiments, the biological sample is independent of samples used to train the classifier.

The classifier may be trained with a first number of independent training samples associated with a presence of the biological state and a second number of independent training samples associated with an absence of the biological state. The first number of independent training samples associated with a presence of the biological state may be no more than the second number of independent training samples associated with an absence of the biological state. The first number of independent training samples associated with a presence of the biological state may be equal to the second number of independent training samples associated with an absence of the biological state. The first number of independent training samples associated with a presence of the biological state may be greater than the second number of independent training samples associated with an absence of the biological state.

The classifier may be configured to identify the biological state with an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%; for at least about 50, at least about 100, at least about 150, at least about 200, at least about 250, at least about 300, or more than about 300 independent samples. The accuracy of identifying the biological state by the classifier may be calculated as the percentage of independent test samples (e.g., subjects having the biological state) that are correctly identified or classified as having or not having the biological state, respectively.

The classifier may be configured to identify the biological state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The PPV of identifying the biological state by the classifier may be calculated as the percentage of biological samples identified or classified as having the biological state that correspond to subjects that truly have the biological state. A PPV may also be referred to as a precision.

The classifier may be configured to identify the biological state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The NPV of identifying the biological state by the classifier may be calculated as the percentage of biological samples identified or classified as not having the biological state that correspond to subjects that truly do not have the biological state.

The classifier may be configured to identify the biological state with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The clinical sensitivity of identifying the biological state by the classifier may be calculated as the percentage of independent test samples associated with presence of the biological state (e.g., subjects known to have the biological state) that are correctly identified or classified as having the biological state. A clinical sensitivity may also be referred to as a recall.

The classifier may be configured to identify the biological state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The clinical specificity of identifying the biological state by the classifier may be calculated as the percentage of independent test samples associated with absence of the biological state (e.g., apparently healthy subjects with negative clinical test results for the biological state) that are correctly identified or classified as not having the biological state.

The classifier may be configured to identify the biological state with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The AUC may be calculated as an integral of the Receiver Operator Characteristic (ROC) curve (e.g., the area under the ROC curve) associated with the classifier in classifying biological samples as having or not having the biological state.

The classifier may be adjusted or tuned to improve the performance, accuracy, PPV, NPV, clinical sensitivity, clinical specificity, or AUC of identifying one or more biological states. The classifier may be adjusted or tuned by adjusting parameters of the classifier (e.g., a set of cutoff values used to classify a sample as described elsewhere herein, or weights of a neural network). The classifier may be adjusted or tuned continuously during the training process or after the training process has completed.

After the classifier is initially trained, a subset of the inputs may be identified as most influential or most important to be included for making high-quality classifications. For example, a subset of the set of nutrigenetic aberrations (e.g., nutrigenetic variants) may be identified as most influential or most important to be included for making high-quality classifications or identifications of the biological state. The set of nutrigenetic variants or a subset thereof may be ranked based on metrics indicative of each nutrigenetic variant's influence or importance toward making high-quality classifications or identifications of the biological state. Such metrics may be used to reduce, in some cases significantly, the number of input variables (e.g., predictor variables) that may be used to train the classifier to a desired performance level (e.g., based on a desired minimum accuracy, PPV, NPV, clinical sensitivity, clinical specificity, or AUC).

For example, if training the training algorithm with a plurality comprising several dozen or hundreds of input variables (e.g., nutrigenetic variants) in the classifier results in an accuracy of classification of more than 99%, then training the training algorithm instead with only a selected subset of 1, 2, 3, 4, 5, 6, 7, 8, 9, 10, no more than about 10, no more than about 15, no more than about 20, no more than about 25, no more than about 30, no more than about 35, no more than about 40, no more than about 45, no more than about 50, or no more than about 100 such most influential or most important input variables (e.g., nutrigenetic variants) among the plurality results in decreased but still acceptable accuracy of classification (e.g., at least about 70%, at least about 75%, at least about 80%, at least about 85%, at least about 90%, at least about 95%, at least about 96%, at least about 97%, or at least about 98%).

In some embodiments, the subset may be selected by rank-ordering the entire plurality of input variables (e.g., nutrigenetic variants) and selecting a predetermined number (e.g., 1, 2, 3, 4, 5, 6, 7, 8, 9, 10, no more than about 10, no more than about 15, no more than about 20, no more than about 25, no more than about 30, no more than about 35, no more than about 40, no more than about 45, no more than about 50, no more than about 100, no more than about 150, or no more than about 200) of input variables with the best metrics. In some embodiments, the selected subset of the influential or most important input variables comprises one or more nutrigenetic aberrations (e.g., nutrigenetic variants) selected from Table 1.

TABLE 1
Nutrigenetic variants

| | RS | SNP | Nucleotide |
|---|---|---|---|
| Gene Name | Number | Identifier | Change |
| ACE | rs4646994 | Ins./Del | I/D |
| ACTN3 | rs1815739 | | 577R > X/ |
| | | | C > T |
| ADIPOQ -11391 | rs17300539 | G −11391A | G > A |
| ADRB2 | rs1042713 | Arg16Gly/G16R | A > G |
| ADRB2 | rs1042714 | Gln27Glu | C > G |
| ADRB3 | rs4994 | Trp64Arg | T > C |
| AGT | rs5051 | −6 G > A | G > A |
| AGT | rs699 | T803C/Met235Thr/ | T > C |
| | | C4072T | |
| APOA2 | rs5082 | −492 T > C | T > C |
| APOA5 | rs662799 | T −1131C | T > C |
| APOC3 | rs5128 | 3175 C > G/ | C > G |
| | | 3238 C > G | |
| APOE | rs429358 | ARG-CYS 112/158 | T > C |
| | | (E2, E3, E4) | |
| APOE | rs7412 | E2/E3/E4 | T > C |
| BDNF | rs6265 | Val66Met, G196A | G > A |
| CAT | rs1001179 | −262 C > T | C > T |
| CETP | rs708272 | Taq1B/279 G > A | G > A |
| CLOCK/ | rs1801260 | 3111 T > C | T > C |
| TMEM165 | | | |
| COL1A1 | rs1800012 | 1546 G > T/G2046T | G > T |
| COL5A1 | rs12722 | BstUI C > T | C > T |
| COMT | rs4680 | Val158Met/ | G > A |
| | | 472 G > A | |
| CRP-3 | rs1205 | 2147 G > A | C > T/G > A |
| CYP17A1 | rs743572 | T −34C | A > G/A > T |
| CYP1A1 | rs4646903 | 6235 T > C/ | T > C |
| | | Msp1 T > C | |
| CYP1A1 | rs1048943 | Ile462Val/+4889A > G/ | A > G |
| | | *2C | |
| CYP1A2 | rs762551 | A −164C/ | A > C |
| | | A −163C/*1F | |
| CYP1B1 | rs1056836 | L432V/1294 C > G/ | C > G |
| | | Val432Leu | |
| DIO2 | rs225014 | Thr92Ala | T > C |
| DRD2/ANNK1 | rs18000497 | Taq1A/2A | C > T |
| eNos | rs1799983 | 984 G > T/ | |
| | | Glu298Asp | |
| EPHX1 | rs1051740 | Tyr113His/113T > C | T > C |
| F2 (Prothrombin | rs1799963 | G20210A | G > A |
| Factor II) | | | |
| F5 (Factor V | rs6025 | R506Q/1691G > A | C > T/G > A |
| Leiden) | | | |
| FABP2 | rs1799883 | Ala54Thr | G > A |
| FTO | rs9939609 | | T > C |
| FUT2 | rs602662 | G772A/Ser258Gly | G > A |
| GDF5 | rs143383 | 5′ UTR C > T | C > T |
| GPX1 | rs1050450 | Pro198Leu | C > T |
| GSTM1 | rs366631 | A 1998G/Ins/del | A > G |
| GSTP1 | rs1695 | Ile105Val/A313G | A > G |
| GSTT1 | rs2266633 | Ins./Del | C > T |
| HFE | rs1799945 | C282Y/His63Asp | C > G |
| HFE | rs1800562 | C282Y and H63D | G > A |
| HO-1 | rs2071746 | +413A > T | A > T |
| hOGG1 | rs1052133 | Ser326Cys | C > G |
| IL-1A-2 | rs17561 | G4845T | G > T |
| IL-1B | rs16944 | A −511G | A > G |
| IL-1B | rs1143634 | 3954 C > T | C > T |
| IL-6 | rs1800795 | G −174C/ | G > C |
| | | C −237G | |
| IL-6R | rs2228145 | 48867 A > C/ | A > C |
| | | Asp358Ala | |
| IL1-RN | rs419598 | 2108C > T | C > T |
| IRS1 | rs2943641 | | C > T |
| LEPR | rs1805094/ | 1968G > C/ | G > C |
| | rs8179183 | Lys656Asn | |
| LEPR-1 | rs1137101 | Gln223Arg/668A > G | A > G |
| LEPR-2 | rs1l37100 | Lys109Arg/K109R | A > G |
| LPL | rs328 | 1595 C > G S447X | C > G |
| MC4R | rs17782313 | | T > C |
| MMP1 | rs1799750 | −1607 1G/2G | delC |
| MMP2 | rs1132896 | Gly226Gly | G > C |
| MnSOD2 | rs4880 | Val16Ala/47T > C | T > C |
| MTHFR-1 | rs1801133 | C677T | C > T or |
| | | | G > A |
| MTHFR-2 | rs1801131 | A1298C | A > C |
| MTR | rs1805087 | A2756G | A > G |
| MTRR | rs1801394 | A −66G | A > G |
| NOS3 | rs1799983 | G984T/Glu298Asp | G > T |
| NQO1 | rs1800566 | C609T/Pro187Ser | C > T |
| NRF2 | rs7181866 | | A > G |
| PAI-1-675 | rs1799889 | 4G/5G | I/D 5G > 4G |
| PLIN1 | rs894160 | 11482 G > A | G > A/C > T |
| PON1 | rs662 | Q192R/Gln > Arg | A > G |
| PPARA | rs4253778 | | G > C |
| PPARG | rs1801282 | Pro12Ala | C > G |
| PPARGC1A | rs8192678 | Gly482Ser | G > A |
| SLC2A2 | | | |
| SLC2A2/GLUT2 | rs5400 | Thr110Ile | C > T |
| SULT1A1 | rs9282861 | Arg213His/638G > A | G > A |
| TCF7L2 | rs7903146 | IVS3C > T | C > T/C > G |
| TIMP4 | rs3755724 | T −55C | T > C |
| TNFA | rs1800629 | G −308A | G > A |
| UCP1 | rs1800592 | −3826 A > G | A > G |
| UCP2 | rs659366 | 866 G > A/Ala55Val/ | G > A |
| | | Ins./Del | |
| UCP3 | rs1800849 | −55 C > T | C > T |
| VDR | rs1544410 | Bsm1 RFLP | G > A |
| VDR | rs2228570 | Fok1 | T > C |
| VDR2 | rs731236 | Taq1 | T > C |
| VEGF | rs2010963 | −634G > C | G > C |

### Computer Systems

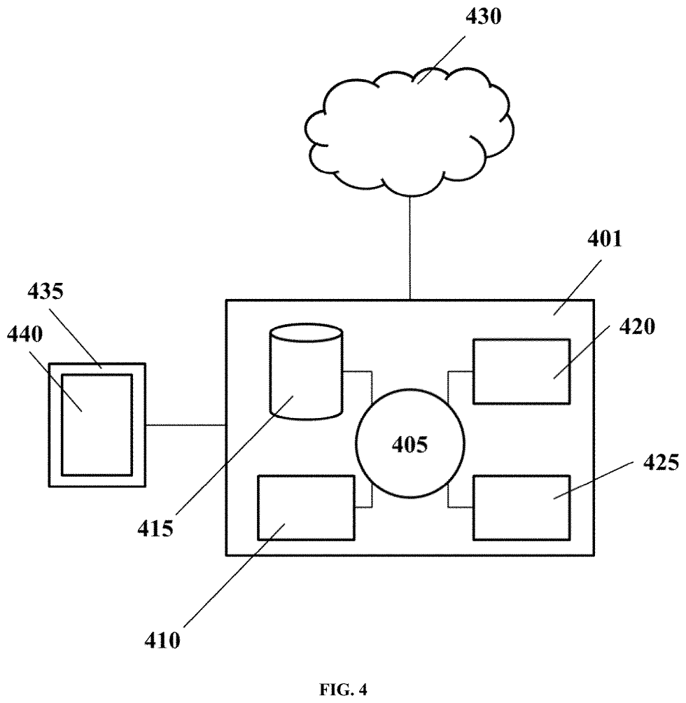

The present disclosure provides computer systems that are programmed to implement methods of the disclosure. FIG. 4 shows a computer system 401 that is programmed or otherwise configured to perform one or more functions or operations for facilitating nutrigenomics reporting for a subject. The computer system 401 can regulate various aspects of the portal and/or platform of the present disclosure, such as, for example, receiving genetic information of a subject comprising a plurality of nucleic acid sequences, obtaining genetic information by processing a biological sample obtained or derived from a subject using one or more nutrigenetic assays, processing genetic information to identify one or more nutrigenetic aberrations (e.g., nutrigenetic variants) of a subject, identifying one or more biological states corresponding to a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an infection pathway, a circadian rhythm pathway, a mood pathway, a memory pathway, a carbohydrate metabolism pathway, an adrenal pathway, a lipid metabolism pathway, a stress pathway, or a cardiovascular health pathway of a subject based at least in part on identified nutrigenetic aberrations (e.g., nutrigenetic variants), outputting an electronic report indicative of one or more biological states of a subject, generating a nutrigenetic health regimen for a subject based at least in part on identified nutrigenetic aberrations (e.g., nutrigenetic variants), transmitting an electronic report to a remote user, storing an electronic report on a remote server, and processing identified nutrigenetic aberrations (e.g., nutrigenetic variants) using a trained algorithm to identify biological states. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet.

The network 430 in some cases is a telecommunication and/or data network. The network 430 can include one or more computer servers, which can enable distributed computing, such as cloud computing. For example, one or more computer servers may enable cloud computing over the network 430 (“the cloud”) to perform various aspects of analysis, calculation, and generation of the present disclosure, such as, for example, receiving genetic information of a subject comprising a plurality of nucleic acid sequences, obtaining genetic information by processing a biological sample obtained or derived from a subject using one or more nutrigenetic assays, processing genetic information to identify one or more nutrigenetic aberrations (e.g., nutrigenetic variants) of a subject, identifying one or more biological states corresponding to a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an infection pathway, a circadian rhythm pathway, a mood pathway, a memory pathway, a carbohydrate metabolism pathway, an adrenal pathway, a lipid metabolism pathway, a stress pathway, or a cardiovascular health pathway of a subject based at least in part on identified nutrigenetic aberrations (e.g., nutrigenetic variants), outputting an electronic report indicative of one or more biological states of a subject, generating a nutrigenetic health regimen for a subject based at least in part on identified nutrigenetic aberrations (e.g., nutrigenetic variants), transmitting an electronic report to a remote user, storing an electronic report on a remote server, and processing identified nutrigenetic aberrations (e.g., nutrigenetic variants) using a trained algorithm to identify biological states. Such cloud computing may be provided by cloud computing platforms such as, for example, Amazon Web Services (AWS), Microsoft Azure, Google Cloud Platform, and IBM cloud. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC). The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user (e.g., a mobile device of the user). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods provided herein can be implemented by way of machine (e.g., computer processor) executable code stored on an electronic storage location of the computer system 401, such as, for example, on the memory 410 or electronic storage unit 415. The machine-executable or machine-readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 401 can include or be in communication with an electronic display 435 that comprises a user interface (UI) 440 for providing, for example, genomic or other data management. Examples of user interfaces include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, receive genetic information of a subject comprising a plurality of nucleic acid sequences, obtain genetic information by processing a biological sample obtained or derived from a subject using one or more nutrigenetic assays, process genetic information to identify one or more nutrigenetic aberrations (e.g., nutrigenetic variants) of a subject, identify one or more biological states corresponding to a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an infection pathway, a circadian rhythm pathway, a mood pathway, a memory pathway, a carbohydrate metabolism pathway, an adrenal pathway, a lipid metabolism pathway, a stress pathway, or a cardiovascular health pathway of a subject based at least in part on identified nutrigenetic aberrations (e.g., nutrigenetic variants), output an electronic report indicative of one or more biological states of a subject, generate a nutrigenetic health regimen for a subject based at least in part on identified nutrigenetic aberrations (e.g., nutrigenetic variants), transmit an electronic report to a remote user, store an electronic report on a remote server, and process identified nutrigenetic aberrations (e.g., nutrigenetic variants) using a trained algorithm to identify biological states.

### EXAMPLES

Example 1

### Methodologies for Quantifying the Scientific Validity and Clinical Utility of Genetic Variants in Metabolic Pathways

Using methods and systems of the present disclosure, a pathway-based nutrigenetic profile of a subject is generated based at least in part on a quantified scientific validity and/or clinical utility of genetic variants (e.g., nutrigenetic variants) in metabolic pathways. First, a methodology is developed to objectively evaluate the scientific validity and clinical utility of genetic variants (including, but not limited to, single nucleotide polymorphisms (SNPs), copy number variations (CNVs), copy number alternations (CNAs), indels (insertions or deletions), gene fusions, translocations, etc.), specifically through a metabolic pathway lens. Next, an impact score is calculated for each SNP genotype per pathway. For example, the impact score may be determined by a static set of scientific and clinical rules as part of the objective evaluation. Next, the genetic information is obtained from a subject is transformed into respective impact scores per pathway as calculated above. For example, the genetic information may be obtained by analyzing a biological sample obtained or derived from the subject using a genetic assay. Next, the genotyping impact scores are utilized as data input for a pathway model and/or algorithm that assigns a biological state to a metabolic pathway.

In some embodiments, the objective evaluation criteria for scientific validity include genotype frequency, study quality and type, biochemical plausibility, and the type of interaction a given SNP has on a pathway. Evaluation for clinical utility may include biochemical impact on clinical dysfunction and/or manifestation, interventions that can modulate the biochemical impact on dysfunction and/or manifestation, measurables, and biomarkers, as well as the probability of benefit from intervention compared to standard guidelines (e.g., how clinical decision making is impacted by the knowledge of the presence of a specific genotype for a specific pathway). In some embodiments, the evaluation criteria for scientific validity and clinical utility for SNP genotypes are scored with a pathway lens, using a scoring rubric with a static set of rules.

Table 2 provides examples of objective evaluation criteria and a static set of rules used to calculate genotype scores per SNP per pathway, based on scientific validity.

TABLE 2
Rules and scores for quantifying scientific validity of genetic variants

| | | | 4. Biological | |
|---|---|---|---|---|
| 1. Genotype | 2. Rating Study | 3. Rating Study | Plausibility | |
| Frequency | Type | Quality | (Biochemistry) | 5. SNP × Pathway |
| Minor Allele | Type of Study and | 3 = Convincing. | 4 = Yes. SNP has a | 4 = Direct |
| Frequencies | Quantity: | Biological | functional effect. | interaction with |
| (MAF): | 3 = Systematic | mechanism of the | Effect is proven in | primary |
| 39% ≤ 4 ≤ 50% | Review or Meta- | interaction is fully | human in vivo | biochemical |
| 31% ≤ 3 < 39% | Analysis. | understood/largely | studies. | pathway. SNP |
| 19% ≤ 2 < 31% | 2 = Randomized | explained. | 3 = Yes, SNP has a | affects an |
| 1% ≤ 1 < 19% | control studies, and | 2 = Probable. | functional effect. | important or main |
| 0 < 1% | Observational | Biological | Effect is proven in | role player in |
| | studies (case | mechanism of the | in vitro studies | pathway. |
| | control, cohort & | interaction is partly | conducted in | 3 = High |
| | case series). | explained. | human cell lines | Intermediate. |
| | Association | 1 = Possible. Only | and animal in vivo | SNP to pathway. |
| | studies. | shown correlation/ | studies. | SNP affects |
| | 1 = Animal or Cell | association. | 2 = Yes. SNP has a | pathway but effect |
| | studies | BONUS point = 3 | functional effect. | is downstream. |
| | BONUS point = 3 | or more | Effect is proven in | 2 = Low |
| | or more | independent studies | in vitro studies | Intermediate. |
| | independent studies | on the SNP. | conducted in | Pathway to |
| | on the SNP. | | mammalian cell | pathway |
| | | | lines and | interaction. SNP |
| | | | recombinant gene | affects role players |
| | | | expression studies. | in a pathway that |
| | | | 1 = Biochemical | interacts with the |
| | | | interaction is | current pathway. |
| | | | hypothesised but | 1 = Indirect |
| | | | not scientifically | interaction. |
| | | | proven | Supporting |
| | | | (association). | function. |
| | | | Protein Modelling | |
| | | | studies. | |

For example, a SNP genotype may be scored based on genotype frequency, as measured by minor allele frequencies (MAF), whereby a score of 4, 3, 2, 1, or 0 is assigned based on the MAF of the SNP genotype belonging to a particular MAF range among a plurality of MAF ranges. The plurality of MAF ranges may include [39%, 50%], [31%, 39%), [19%, 31%), [1%, 19%), and [0%, 1%).

As another example, a SNP genotype may be scored based on a rating of the study type (e.g., type of study and quantity), whereby a score of 3, 2, or 1 is assigned based on the type and quantity of study that was performed on the SNP genotype (e.g., 3 points for a Systematic Review or Meta-Analysis; 2 points for Randomized control studies, Observational studies (case control, cohort & case series), or Association studies; 1 point for Animal or Cell studies; and a bonus point for having 3 or more independent studies on the SNP).

As another example, a SNP genotype may be scored based on a rating of the study quality, whereby a score of 3, 2, or 1 is assigned based on the quality of study that was performed on the SNP genotype (e.g., 3 points for a convincing study, where the biological mechanism of the interaction is fully understood or largely explained; 2 points for a study of probable quality, where the biological mechanism of the interaction is partly explained; 1 point for a study of possible quality, where the study only shows correlation or association, and a bonus point for having 3 or more independent studies on the SNP).

As another example, a SNP genotype may be scored based on the biological plausibility of the SNP, given its biochemistry. A score of 4, 3, 2, or 1 is assigned based on the biological plausibility of the SNP genotype (e.g., 4 points when the SNP has a functional effect, and the effect is proven in human in vivo studies; 3 points when the SNP has a functional effect, and the effect is proven in in vitro studies conducted in human cell lines and animal in vivo studies; 2 points when the SNP has a functional effect, and the effect is proven in in vitro studies conducted in mammalian cell lines and recombinant gene expression studies; and 1 point when the biochemical interaction is hypothesized but not scientifically proven (e.g., association), such as protein modeling studies).

As another example, a SNP genotype may be scored based on the interaction with a pathway of the SNP. A score of 4, 3, 2, or 1 is assigned based on the pathway of the SNP genotype (e.g., 4 points when there is a direct interaction with a primary biochemical pathway, and the SNP affects an important or main role player in pathway; 3 points when there is a high intermediate SNP-to-pathway interaction, and the SNP affects the pathway, but the effect is downstream; 2 points when there is a low intermediate pathway-to-pathway interaction, and the SNP affects role players in a pathway that interacts with the current pathway; and 1 point when there is indirect interaction, and the SNP has a supporting function).

In addition, Table 3 provides examples of objective evaluation criteria and a static set of rules used to calculate genotype scores per SNP per pathway, based on clinical utility.

TABLE 3
Rules and scores for clinical validity of genetic variants

| | | | D. Probability of |
|---|---|---|---|
| A. Biochemical impact | B. Is there an | | benefit from |
| on clinical dysfunction/ | intervention (E) that | C. Measurables and | intervention compared |
| manifestation | can modulate A? | Biomarkers | to standard guideline |
| 4 = SNP is directly | 4 = Proven significant | 4 = Direct measure of | 4 = Clear change to E. |
| implicated in clinical | interaction between the | SNP's effect. | Definitive change from |
| phenotype. | SNP, intervention, and | Name of biomarker is the | standard interventions. |
| 3 = Gene. Protein, or | phenotype. | same as gene. | Guides practitioner to |
| Enzyme implicated in | Intervention linked | 3 = Indirect measure of | make better informed |
| clinical phenotype. | directly to SNP. | SNP's functional effect | recommendations in |
| 2 = Pathway is | 3 = Intervention impact | Downstream tag in | comparison to standard |
| implicated in clinical | on gene, protein, or | cascade. | guidelines. |
| phenotype. | enzyme. | 2 = Measure the | 3 = Change focus of |
| 1 = Theoretical. | 2 = Pathway-based | pathway. End product of | standard interventions. |
| Biochemical rationale | intervention. | pathway. | 2 = Minor adjustments to |
| can be justified. | 1 = Theoretical. | 1 = Theoretical. No test | standard guidelines. |
| | Biochemical rationale | available, but there | 1 = Make no difference. |
| | can be justified. | should be one. | Standard guidelines still |
| | | Biochemical rationale | applied. |
| | | can be justified. | |

As an example, a SNP genotype may be scored based on a rating of the biochemical impact of the SNP on clinical dysfunction and/or manifestation type, whereby a score of 4, 3, 2, or 1 is assigned based on the biochemical impact of the SNP (e.g., 4 points when the SNP is directly implicated in clinical phenotype; 3 points when the gene, protein, or enzyme associated with the SNP is implicated in a clinical phenotype; 2 points when the pathway of the SNP is implicated in the clinical phenotype; and 1 point when the SNP has a theoretical biochemical impact, and a biochemical rationale can be justified).

As another example, a SNP genotype may be scored based on whether there is an intervention (E) that can modulate the biochemical impact (A), whereby a score of 4, 3, 2, or 1 is assigned based on the existence of an intervention for the SNP's biochemical impact (e.g., 4 points when there is a proven significant interaction between the SNP, the intervention, and the clinical phenotype, and the intervention is linked directly to the SNP; 3 points when the intervention has an impact on the gene, protein or enzyme associated with the SNP; 2 points when there is a pathway-based intervention for the SNP's biochemical impact; and 1 point when there is a theoretical pathway-based intervention for the SNP's biochemical pathway, and a biochemical rationale can be justified).

As another example, a SNP genotype may be scored based on measurables and biomarkers of the SNP (e.g., the SNP's functional effects), whereby a score of 4, 3, 2, or 1 is assigned based on the measurables and biomarkers (e.g., 4 points when there is a direct measure of the SNP's effect, and the name of the biomarker is the same as the gene; 3 points when there is an indirect measure of the SNP's functional effect, and there is a downstream tag in the cascade; 2 points when the pathway has been measured, and the end product of the pathway is measurable; and 1 point when the SNP's effect is theoretical, such that no test available, but there should be one, and a biochemical rationale can be justified).

As another example, a SNP genotype may be scored based on a probability of a benefit to a subject from intervention compared to a standard guideline, whereby a score of 4, 3, 2, or 1 is assigned based on the probability of benefit from intervention (e.g., 4 points when there is a clear change to (E), such that a definitive change from standard interventions exists and can guide practitioners to make better informed recommendations in comparison to standard guidelines; 3 points when an intervention can be used to change the focus of standard interventions; 2 points when an intervention can be used to make minor adjustments to standard guidelines; and 1 point when the intervention makes no difference, and standard guidelines are still applied.

In addition, Table 4 provides additional SNPs added to the genotyping panel, which may be used in conjunction with methods and systems of the present disclosure.

TABLE 4
Additional SNPs added to genotyping panel

| GENE | RS | SNP | NUCLEOTIDE |
|---|---|---|---|
| NAME | NUMBER | IDENTIFIER | CHANGE |
| HTR1A | rs6295 | −1019 C > G | C> |
| ACE | rs4343 | G2350A | G > A |
| ADIPOQ | rs17366568 | | G > A |
| AKT1 | rs2494732 | G1172 +23A | T > C |
| ALDH2 | rs671 | Glu457Lys or | G > A |
| | | G1369A | |
| ANK3 | rs10994336 | | C > T |
| ANK3 | rs1938526 | | A > G |
| AOC1/DAO | rs1049793 | His645Asp/ | C > G |
| | | 1933C > G | |
| APOA5-A4-C3-A1 | rs12272004 | Near APOA5 gene | C > A |
| BCMO1 | rs7501331 | A379V/C1136T | C > T |
| BCMO1 | rs12934922 | R267S/Arg267Ser | A > G/A > T |
| BHMT | rs3733890 | G742A | G > A |
| BHMT | rs651852 | Arg239Gln +742G > A | G > A/C > T |
| CACNA1C | rs1006737 | | G > A |
| CBS | rs234706 | C699T | C > T/G > A |
| CETP | rs247616 | 279G > A (C > T) | G > A/C > T |
| CHRNA5 | rs16969968 | D398N | G > A |
| CHRNA5 | rs951266 | Asp398Asn | G > A |
| | | D398N | |
| CNR1 | rs2023239 | | T > C |
| CYP1B1 | rs1800440 | Asn453Ser/N453S | A > G |
| CYP2C19 | rs12248560 | *1/*17 −806 C > T | C > A/C > T |
| CYP2C19 | rs4244285 | 681G > A | G > A |
| CYP2C19 | rs4986893 | Trp212Ter | G > A |
| CYP2C9 | rs1057910 | Ile359Leu | A > C |
| | | A1075C | |
| CYP2C9 | rs1799853 | Arg144Cys/C430T | C > T |
| CYP2D6*10 | rs1065852 | 100C > T | C > T |
| | | CYP2D6 *10 | |
| | | Pro34Ser | |
| | | P34S | |
| CYP2D6*2 | rs16947 | Arg296Cys/R296C | G > A |
| | | CYP2D6*2 | |
| CYP2D6/CYP2D6*17 | rs28371706 | T107I/1023C > T | G > A/G > T |
| CYP2D6*3A | rs35742686 | 2549delA | delT |
| CYP2D6M | rs3892097 | 1846G > A/*1/*4 | G > A |
| CYP2D6*6 | rs5030655 | 1707delT | delA |
| CYP2R1 | rs10741657 | | G > A |
| CYP3A4/CYP3A4*1B | rs2740574 | A −392G or *1/*1 | A > G |
| DAO (APB1 or AOC1) | rs10156191 | C47T/Thr16Met | C > T |
| DAO (APB1 or AOC1) | rs1049742 | Ser332Phe/995C > T | C > T |
| DRD1 | rs4532 | −48G > A | G > A/C > T |
| DRD1 | rs5326 | | C > T |
| | | | G > A |
| DRD3 | rs6280 | Ser9Gly | C > T |
| DRD4 | rs1800955 | C −521T | C > T |
| FAAH | rs324420 | 385C > A/Pro129Thr | C > A |
| FADS1 | rs174537 | 592 G > T | G > T |
| FKBP2 | rs11607007 | | C > T |
| FOXO1 | rs10507486 | | G > A |
| FOXO1 | rs2297627 | | A > G |
| | | | T > C |
| FOXO3 | rs2802292 | | G > T |
| FTO | rs1121980 | | C > T |
| FTO | rs8050136 | | C > A |
| FUT2 | rs601338 | G428A/Trp154Ter | G > A |
| GABRA2 | rs279858 | 396A > G | A > G |
| | | | T > C |
| GC | rs2282679 | | T > G, A > C |
| GCLM | rs3170633 | | C > T |
| GCLM | rs2301022 | −588C > T | C > T |
| GSK3B | rs11925868 | | A > C |
| GSK3B | rs11927974 | | G > A |
| GSK3B | rs334555 | | C > G |
| GSTP1 | rs1138272 | A114V/C341T | C > T |
| HLA-DQ2.2 | rs2395182 | | G > T |
| HLA-DQ8 | rs7454108 | | T > C |
| HLA-DQA1/HLA- | rs2187668 | | G > A |
| DQ2.5 | | | |
| HMNT | rs1050891 | 939A > G | A > G |
| HMNT | rs1801105 | 314C > T/T105I | C > T |
| | Merged into | | |
| | rs11558538 | | |
| LCT (or MCM6) | rs4988235 | −13910 C > T | C > T |
| MAOA | rs909525 | C42795T | A > G |
| MTHFD1 | rs2236225 | G1958A | G > A |
| NAT1/ | rs4986782 | Arg187Gln/R187Q | G > A |
| NAT1*14B | | | |
| NAT2 | rs1495741 | 62 G > A | G > A |
| NAT2 | rs1799930 | G590A/Arg197Gln | G > A |
| NAT2 | rs1799931 | G857A/Gly286Glu | G > A |
| | | NAT2*7B | |
| NAT2 | rs1801279 | G191A/Arg64Gln | G > A |
| | | | G > A(*14) |
| NAT2 | rs1801280 | 341T > C/Ile114Thr | Paired SNP T > C |
| | | NAT2*5A | |
| NBPF3/ | rs4654748 | | C > T |
| ALPL | | | |
| OPRMI | rs1799971 | Asn40Asp/118 A > G | A > G |
| OXTR | rs53576 | | G > A |
| PEMT | rs12325817 | | G > C |
| PEMT | rs7946 | G5465A | G > A/C > T |
| SHMT1 | rs1979277 | C1420T | G > A |
| | | Leu474Phe | C > T |
| SIRT1 | rs12413112 | | G > A |
| SIRT1 | rs12778366 | | T > C/T > G |
| SIRT1 | rs2273773 | 994T > C | T > C |
| SIRT1 | rs3740051 | | A > G/A > T |
| SIRT1 | rs3758391 | | T > C |
| SLC23A1 | rs33972313 | G790A | G > A |
| SLC6A4 | rs1042173 | c. *463T > G | T > G/A > C |
| SLCO1B1 | rs4149056 | Val174Ala or 521T > C | T > C |
| TAS2R38 | rs1726866 | 785T > C/Val262Ala | T > C |
| | | | G > A |
| TCN2 | rs1801198 | C776G | C > G |
| | | Pro259Arg | |
| TERT | rs2736098 | 915G > A | C > T/G > A |
| VKORC1 | rs9923231 | −1639G > A | G > A |
| APC | rs1801155 | I1307K | T > A |
| TF | rs3811647 | | G > A |

A set of impact scores, grouped as part of the same metabolic pathway, may be summed and expressed as a percentage of the total pathway score to assign a pathway weighting, with or without a pre-adjusted weighting for the clinical scoring component. Boundaries of biological states may be determined by calculating the maximum probable pathway score from a set of genotyping scores and set it as upper limit. The lower limit may be set as the minimum probable pathway score from a set of pathway scores. The range between the upper and lower limit may then be used to inform and/or calculate the boundary thresholds, taking into account the number of biological states. A regression model may be applied to examine the effect of calculated boundary thresholds on pathway states. Classification thresholds (specific to a pathway) to determine the boundaries of different biological states may also be calculated by cluster analysis, for example, but not limited to, k-means clustering or principal component analysis.

A subject's genetic information obtained from a genetic assay is transformed to assign an impact score per SNP genotype per pathway. This serves as input for a pathway-specific model and/or algorithm (derived from models described in the previous paragraph) that classifies a subject's pathway score into a biological state for that pathway. A clinical translation layer is coupled to each biological state, and may comprise of clinical recommendations for lifestyle, diet and supplements, specific to that pathway.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-102. (canceled)

103. A computer-implemented method for generating a nutrigenetic profile of a subject, the method comprising:
(a) receiving genetic information of said subject, which genetic information comprises a plurality of nucleic acid sequences, wherein said genetic information is obtained by processing a biological sample obtained or derived from said subject using one or more nutrigenetic assays;
(b) processing said genetic information to identify one or more nutrigenetic aberrations of said subject;
(c) identifying one or more biological states corresponding to at least one of a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an infection pathway, a circadian rhythm pathway, a mood pathway, a memory pathway, a carbohydrate metabolism pathway, an adrenal pathway, a lipid metabolism pathway, a stress pathway, and a cardiovascular health pathway of said subject based at least in part on said one or more nutrigenetic aberrations identified in (b); and
(d) outputting an electronic report indicative of said one or more biological states of said subject.

104. The method of claim 103, wherein said plurality of nucleic acid sequences comprises deoxyribonucleic acid (DNA) sequences, ribonucleic acid (RNA) sequences, or a combination thereof.

105. The method of claim 103, wherein said biological sample is selected from the group consisting of saliva, cheek swab, blood, plasma, serum, urine, and a combination thereof.

106. The method of claim 103, wherein said one or more nutrigenetic assays comprise one or more of: a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk, a migraine test, a thyroid test, an eczema test, and a cancer genetics assay.

107. The method of claim 106, wherein said one or more nutrigenetic assays comprise two or more of: a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk, a migraine test, a thyroid test, an eczema test, and a cancer genetics assay.

108. The method of claim 107, wherein said one or more nutrigenetic assays comprise three or more a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk, a migraine test, a thyroid test, an eczema test, and a cancer genetics assay.

109. The method of claim 103, wherein said one or more nutrigenetic aberrations comprise one or more of: single nucleotide polymorphisms (SNPs), copy number variants (CNVs), insertions or deletions (indels), fusions, and translocations.

110. The method of claim 103, wherein said one or more nutrigenetic aberrations comprise one or more of the nutrigenetic variants in Table 1 or Table 4.

111. The method of claim 103, wherein said cellular pathway comprises one or more of: detoxification, DNA damage, inflammation, methylation, and oxidative stress.

112. The method of claim 103, wherein said functional systems pathway comprises one or more of: blood clotting, bone health, collagen and joints, brain health, glucose and insulin, sex hormone balance, vascular health, cognitive decline, memory loss, mood disorders and behavior, female sex hormone balance, male sex hormone balance, and histamine tolerance.

113. The method of claim 103, wherein said energy pathway comprises one or more of: adipogenesis, appetite/satiety/intake, energy expenditure, exercise response, pro-inflammatory fat, weight gain and weight loss resistance, circadian rhythm, and starch metabolism.

114. The method of claim 103, wherein said activity pathway comprises one or more of: training response (VO2max), endurance, injury, power, recovery, flexibility and strength.

115. The method of claim 103, wherein said nutrients pathway comprises one or more of: caffeine, salt, vitamin D, fatty acids, vitamin A, vitamin B6, vitamin B12, folate, vitamin C, vitamin E, choline, iron overload, iron status, gluten, celiac, and cannaboid metabolism.

116. The method of claim 103, wherein said cardiovascular health pathway comprises one or more of: blood clotting, blood pressure, vascular health, and lipid metabolism.

117. The method of claim 103, wherein (c) further comprises identifying said one or more biological states of said subject based at least in part on one or more of: a diagnosis of a disease or disorder, a prognosis of a disease or disorder, a risk of having a disease or disorder, a treatment history of a disease or disorder, a history of previous treatment for a disease or disorder, a history of prescribed medications, a history of prescribed medical devices, age, height, weight, sex, smoking status, and one or more symptoms of said subject.

118. The method of claim 103, further comprising generating a nutrigenetic health regimen for said subject based at least in part on said identified one or more nutrigenetic aberrations.

119. The method of claim 118, wherein said nutrigenetic health regimen comprises recommendations to prevent onset of a disease or disorder, delay onset of a disease or disorder, reverse a disease or disorder, or maintain a physiological or health state of said subject.

120. The method of claim 118, wherein said nutrigenetic health regimen comprises recommendations related to one or more of: diet, exercise, sports training, supplements, functional tests, blood tests, brain management, behavior change, skin care, environmental exposure, stress management, and mental health.

121. The method of claim 118, wherein said electronic report is indicative of said nutrigenetic health regimen.

122. The method of claim 121, wherein said electronic report is presented on a graphical user interface of an electronic device of a user.

123. The method of claim 103, wherein (c) comprises processing said identified one or more nutrigenetic aberrations using a trained algorithm to identify said one or more biological states.

124. The method of claim 103, wherein said electronic report comprises one or more graphical depictions of said one or more biological states of said subject.

125. The method of claim 103, further comprising using said electronic report to provide said subject with a therapeutic intervention.

126. The method of claim 103, further comprising determining an impact score for each of said one or more nutrigenetic aberrations.

127. The method of claim 126, wherein said impact score for a given nutrigenetic aberration of said one or more nutrigenetic aberrations is determined based at least in part on a scientific validity of said given nutrigenetic aberration or a clinical validity of said given nutrigenetic aberration.

128. The method of claim 127, wherein determining said impact score for a given nutrigenetic aberration of said one or more nutrigenetic aberrations based on said clinical validity comprises determining said impact score based on one or more of biochemical impact of said given nutrigenetic aberration on clinical dysfunction or manifestation, existence of an intervention that modulates said biochemical impact, measurables and biomarkers of said given nutrigenetic aberration, and probability of benefit from said intervention.

129. The method of claim 128, wherein said impact score for a given nutrigenetic aberration of said one or more nutrigenetic aberrations is determined based on said clinical validity according to the rules and scores listed in Table 4.

130. A system for generating a nutrigenetic profile of a subject, comprising:
a database configured to store genetic information of said subject, which genetic information comprises a plurality of nucleic acid sequences, wherein said genetic information is obtained by processing a biological sample obtained or derived from said subject using one or more nutrigenetic assays; and
one or more computer processors operatively coupled to said database, wherein said one or more computer processors are individually or collectively programmed to:
(i) process said genetic information to identify one or more nutrigenetic aberrations of said subject;
(ii) identify one or more biological states corresponding to at least one of a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an infection pathway, a circadian rhythm pathway, a mood pathway, a memory pathway, a carbohydrate metabolism pathway, an adrenal pathway, a lipid metabolism pathway, a stress pathway, and a cardiovascular health pathway of said subject based at least in part on said one or more nutrigenetic aberrations identified in (i); and
(iii) electronically output a report indicative of said one or more biological states of said subject.

131. A non-transitory computer-readable medium comprising machine-executable code that, upon execution by one or more computer processors, implements a method for generating a nutrigenetic profile of a subject, the method comprising:
(a) receiving genetic information of said subject, which genetic information comprises a plurality of nucleic acid sequences, wherein said genetic information is obtained by processing a biological sample obtained or derived from said subject using one or more nutrigenetic assays;
(b) processing said genetic information to identify one or more nutrigenetic aberrations of said subject;
(c) identifying one or more biological states corresponding to at least one of a metabolic pathway, a cellular pathway, a functional systems pathway, an energy pathway, an activity pathway, a nutrients pathway, a skin pathway, an immune pathway, a gut pathway, a thyroid pathway, a mitochondria health pathway, an infection pathway, a circadian rhythm pathway, a mood pathway, a memory pathway, a carbohydrate metabolism pathway, an adrenal pathway, a lipid metabolism pathway, a stress pathway, and a cardiovascular health pathway of said subject based at least in part on said one or more nutrigenetic aberrations identified in (b); and
(d) outputting an electronic report indicative of said one or more biological states of said subject.
